(19) United States
(12) Patent Application Publication
(10) Pub. No.: US 2017/0327494 A1
(43) Pub. Date: Nov. 16, 2017

(54) NON-INVASIVE VENTILATION WITH HIGH FREQUENCY OSCILLATIONS

(71) Applicant: KONINKLIJKE PHILIPS N.V., EINDHOVEN (NL)

(72) Inventors: ROBERT ROMANO, PITTSBURGH, PA (US); PETER DOUGLAS HILL, MONROEVILLE, PA (US)

(21) Appl. No.: 15/526,055

(22) PCT Filed: Nov. 19, 2015
(86) PCT No.: PCT/EP2015/058966

§ 371 Date: May 11, 2017

Related U.S. Application Data
(60) Provisional application No. 62/082,302, filed on Nov. 20, 2014.

(30) Foreign Application Priority Data
Apr. 25, 2014 (IN) 1133/DEL/2014

(51) Int. Cl. C07D 417/14; C07D 409/14; C07D 405/14; A01N 43/82; A01N 43/50; A01N 43/78; A01N 43/76; A01N 43/56; C07D 413/14; C07D 403/14

(52) U.S. Cl. CPC C07D 417/14; C07D 413/14; C07D 409/14; C07D 405/14; C07D 403/14; A01N 43/50; A01N 43/78; A01N 43/76; A01N 43/56; A01N 43/82

(57) ABSTRACT

Compounds of the formula (I) wherein X and Y are independently O, S or NR⁵, R¹, R² and R⁴ are independently hydrogen, halogen, C₁-C₆alkyl, C₁-C₆haloalkyl, C₁-C₆alkoxy or C₁-C₆haloalkoxy and R³ is an aromatic five-membered heterocycle, which can be substituted are useful as a pesticides especially fungicide.

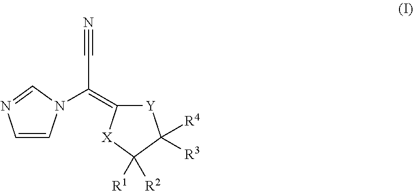

The present invention relates to microbiocidal imidazole derivatives, e.g. as active ingredients, which have microbiocidal activity, in particular fungicidal activity. The invention also relates to preparation of these imidazole derivatives, to imidazole derivatives used as intermediates in the preparation of these imidazole derivatives, to preparation of these intermediates, to agrochemical compositions which comprise at least one of the imidazole derivatives, to preparation of these compositions and to use of the imidazole derivatives or compositions in agriculture or horticulture for controlling or preventing infestation of plants, harvested crops, seeds or non-living materials by phytopathogenic microorganisms, preferably fungi.

The present invention provides compounds of formula I:

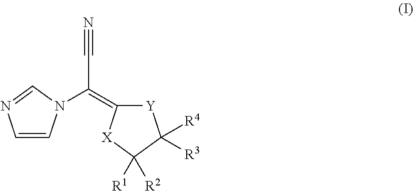

wherein,
X and Y are independently O, S or NR⁵;
R¹, R² and R⁴ are independently hydrogen, halogen, C₁-C₆alkyl, C₁-C₆haloalkyl, C₁-C₆alkoxy or C₁-C₆haloalkoxy;
R³ is an aromatic five-membered heterocycle, and the aromatic five-membered heterocyclic group contains 1 to 4 hetero atoms selected from the group consisting of nitrogen, oxygen and sulfur, it not being possible for each ring system to contain more than 2 oxygen atoms and more than 2 sulfur atoms which and the aromatic five-membered heterocyclic group can be substituted by 1 to 3 substituents selected from halogen, cyano, hydroxyl, amino, nitro, C₁-C₆alkyl, C₁-C₆haloalkyl, C₃-C₆cycloalkyl, C₃-C₆halocycloalkyl, C₁-C₆alkoxy, C₁-C₆haloalkoxy, C₁-C₆alkylthio, C₁-C₆alkylsulfinyl, C₁-C₆alkylsulfonyl, C₁-C₆alkylcarbonyl, C₂-C₆alkenyl, C₂-C₆haloalkenyl, C₂-C₆alkenyloxy, C₂-C₆haloalkenyloxy, C₂-C₆alkynyl, C₂-C₆alkynyloxy, unsubstituted aryl or aryl substituted by 1 to 3 substituents R⁶, unsubstituted heteroaryl or heteroaryl substituted by 1 to 3 substituents R⁶, unsubstituted aryloxy or aryloxy substituted by 1 to 3 substituents R⁶, unsubstituted heteroaryloxy or heteroaryloxy substituted by 1 to 3 substituents R⁶, unsubstituted arylC₁-C₆alkyl or heteroarylC₁-C₆alkyl substituted by 1 to 3 substituents R⁶;
and which can be annulated by a saturated, partially unsaturated or aromatic isocyclic or heterocyclic 6 membered ring to form a bicyclic ring system;
R⁵ is hydrogen, C₁-C₆alkyl, C₁-C₆alkoxy or C₃-C₆cycloalkyl;
R⁶ is independently hydroxyl, amino, nitro, cyano, halogen, C₁-C₆alkyl, C₁-C₆alkoxy, C₁-C₆haloalkyl, C₁-C₆haloalkoxy, aryl, aryloxy or arylC₁-C₆alkyl, in which the alkyl, alkoxy and aryl groups each can be independently substituted by halogen, cyano, C₁-C₆alkyl, C₁-C₆haloalkyl, C₁-C₆alkoxy or C₁-C₆haloalkoxy;
or a salt or a N-oxide thereof.

Where substituents are indicated as being optionally substituted, this means that they can or can't carry one or more identical or different substituents, e.g. one to three substituents. Normally not more than three such optional substituents are present at the same time. Where a group is indicated as being substituted, e.g. alkyl, this includes those groups that are part of other groups, e.g. the alkyl in alkylthio or alkoxy. The same applies for the cycloalkyl, aryl, alkenyl, alkynyl and heteroaryl groups.

The number of substituents does not exceed the number of available C—H and N—H bonds, for example in the group C₁-C₈alkylsulfinyl substituted by one or more R⁶ has only one to three substituents if C₁alkylsulfinyl thus methylsulfinyl is meant.

The term “halogen” refers to fluorine, chlorine, bromine or iodine, preferably fluorine and chlorin.

Alkyl substituents may be straight-chained or branched. Alkyl on its own or as part of another substituent is, depending upon the number of carbon atoms mentioned, for example, methyl, ethyl, n-propyl, n-butyl, n-pentyl, n-hexyl and the isomers thereof, for example, iso-propyl, iso-butyl, sec-butyl, tert-butyl or iso-amyl.

Cycloalkyl includes cyclopropyl, cyclobutyl, cyclopentyl and cyclohexyl.

Alkenyl substituents can be in the form of straight or branched chains, and the alkenyl moieties, where appropriate, can be of either the (E)- or (Z)-configuration. Examples are vinyl and allyl. The alkenyl groups are preferably C₂-C₆, more preferably C₂-C₄ and most preferably C₂-C₃ alkenyl groups.

Alkynyl substituents can be in the form of straight or branched chains. Examples are ethynyl and propargyl. The alkynyl groups are preferably C₂-C₆, more preferably C₂-C₄ and most preferably C₂-C₃ alkynyl groups.

Haloalkyl groups may contain one or more identical or different halogen atoms and, for example, may stand for CH₂Cl, CHCl₂, CCl₃, CH₂F, CHF₂, CF₃, CF₃CH₂, CH₃CF₂, CF₃CF₂ or CCl₃CCl₂.

Alkoxy means a radical —OR, where R is alkyl, e.g. as defined above. Alkoxy groups include, but are not limited to, methoxy, ethoxy, 1-methylethoxy, propoxy, butoxy, 1-methylpropoxy and 2-methylpropoxy.

Haloalkoxy means a radical —OR, where R is haloalkyl, e.g. is described above. Haloalkloxy groups include, but are not limited to, CH₂ClO, CHCl₂O, CCl₃O, CH₂FO, CHF₂O—, CF₃O—, CF₃CH₂O—, CH₃CF₂O or CCl₃CCl₂O—.

Cyano means a —CN group.

Amino means an —NH₂ group.

Hydroxyl or hydroxy stands for a —OH group.

Aryl means a ring system which may be mono-, bi-cyclic. Examples of such rings include phenyl or naphthalenyl. A preferred aryl group is phenyl.

Heteroaryl stands for aromatic heterocyclic ring systems, which can be mono- or bi-cyclic and wherein at least one oxygen, nitrogen or sulfur atom is present as a ring member, which can be accompanied by other oxygen, nitrogen, sulphur atoms as ring members. Monocyclic and bicyclic aromatic ring systems are preferred. For example, monocyclic heteroaryl may be a 5- or 6-membered ring containing one to three heteroatoms selected from oxygen, nitrogen and sulfur, more preferably selected from nitrogen and sulfur. Bicyclic heteroaryl may be a 9- or 10-membered bicyclic ring containing one to five heteroatoms, preferably one to three heteroatoms, selected from oxygen, nitrogen and sulfur. Examples of heteroaryl are furyl, thienyl, pyrrolyl, imidazolyl, pyrazolyl, thiazolyl, isothiazolyl, oxazolyl, isoxazolyl, oxadiazolyl, thiadiazolyl, triazolyl, tetrazolyl, pyridyl, pyridazinyl, pyrimidinyl, pyrazinyl, triazinyl, tetrazinyl, indolyl, benzothiophenyl, benzofuranyl, benzimidazolyl, indazolyl, benzotriazolyl, benzothiazolyl, benzoxazolyl, quinolinyl, isoquinolinyl, phthalazinyl, quinoxalinyl, quinazolinyl, cinnolinyl and naphthyridinyl. Heteroaryl rings do not contain adjacent oxygen ring atoms, adjacent sulfur ring atoms or adjacent oxygen and sulfur ring atoms.

The presence of one or more possible asymmetric carbon atoms in a compound of formula (I) means that the compounds may occur in optically isomeric forms, i.e. enantiomeric or diastereomeric forms. Also atropisomers may occur as a result of restricted rotation about a single bond. Formula (I) is intended to include all those possible isomeric forms and mixtures thereof. The present invention includes all those possible isomeric forms and mixtures thereof for a compound of formula I. Likewise, formula (I) is intended to include all possible tautomers. The present invention includes all possible tautomeric forms for a compound of formula I.

In each case, the compounds of formula (I) according to the invention are in free form, in oxidized form as a N-oxide or in salt form, e.g. an agronomically usable salt form.

N-oxides are oxidized forms of tertiary amines or oxidized forms of nitrogen containing heteroaromatic compounds. They are described for instance in the book “Heterocyclic N-oxides” by A. Albini and S. Pietra, CRC Press, Boca Raton 1991.

The term “fungicide” as used herein means a compound that controls, modifies, or prevents the growth of fungi. The term “fungicidally effective amount” means the quantity of such a compound or combination of such compounds that is capable of producing an effect on the growth of fungi. Controlling or modifying effects include all deviation from natural development, such as killing, retardation and the like, and prevention includes barrier or other defensive formation in or on a plant to prevent fungal infection.

The term “herbicide” as used herein means a compound that controls or modifies the growth of plants.

The term “herbicidally effective amount” means the quantity of such a compound or combination of such compounds that is capable of producing a controlling or modifying effect on the growth of plants. Controlling or modifying effects include all deviation from natural development, for example killing, retardation, leaf burn, albinism, dwarfing and the like.

The term “insecticide” as used herein means a compound that controls or modifies the growth of insects. The term “insecticidally effective amount” means the quantity of such a compound or combination of such compounds that is capable of killing, controlling, or infecting insects, retarding the growth or reproduction of insects, reducing an insect population, and/or reducing damage to plants caused by insects.

The term “locus” as used herein means fields in or on which plants are growing, or where seeds of cultivated plants are sown, or where seed will be placed into the soil. It includes soil, seeds, and seedlings, as well as established vegetation.

The term “metabolism” as used herein means the conversion or breakdown of a substance from one form to another by a living organism.

The term “nematicide” as used herein means a compound that controls or modifies the growth of nematodes. The term “nematicidally effective amount” means the quantity of such a compound or combination of such compounds that is capable of killing, controlling, or infecting nematodes, retarding the growth or reproduction of nematodes, reducing a nematode population, and/or reducing damage to plants caused by nematodes.

A nematicidally effective amount” as used herein refers to an amount of nematicide capable of killing, controlling, or infecting nematodes, retarding the growth or reproduction of nematodes, reducing a nematode population, and/or reducing damage to plants caused by nematodes

The term “plants” refers to all physical parts of a plant, including seeds, seedlings, saplings, roots, tubers, stems, stalks, foliage, and fruits.

The following list provides definitions, including preferred definitions, for substituents R¹, R², R³, R⁴, R⁵, R⁶, R⁷, X and Y with reference to compounds of formula (I). For any one of these substituents, any of the definitions given below may be combined with any definition of any other substituent given below or elsewhere in this document.

X and Y are independently O, S or NR⁵.

Preferably X and Y are independently O or S, more preferably S.

R¹, R² and R⁴ are independently hydrogen, halogen, C₁-C₆alkyl, C₁-C₆haloalkyl, C₁-C₆alkoxy or C₁-C₆haloalkoxy.

Preferably R¹, R² and R⁴ are independently hydrogen, halogen, C₁-C₆alkyl or C₁-C₆haloalkyl; more preferably hydrogen or C₁-C₆alkyl; even more preferably hydrogen or methyl.

R³ is an aromatic five-membered heterocycle, and the aromatic five-membered heterocyclic group contains 1 to 4 hetero atoms selected from the group consisting of nitrogen, oxygen and sulfur, it not being possible for each ring system to contain more than 2 oxygen atoms and more than 2 sulfur atoms which and the aromatic five-membered heterocyclic group can be substituted by halogen, cyano, hydroxyl, amino, nitro, C₁-C₆alkyl, C₁-C₆haloalkyl, C₃-C₆cycloalkyl, C₃-C₆halocycloalkyl, C₁-C₆alkoxy, C₁-C₆haloalkoxy, C₁-C₆alkylthio, C₁-C₆alkylsulfinyl, C₁-C₆alkylsulfonyl, C₁-C₆alkylcarbonyl, C₂-C₆alkenyl, C₂-C₆haloalkenyl, C₂-C₆alkenyloxy, C₂-C₆haloalkenyloxy, C₂-C₆alkynyl, C₂-C₆alkynyloxy, unsubstituted aryl or aryl substituted by 1 to 3 substituents R⁶, unsubstituted heteroaryl or heteroaryl substituted by 1 to 3 substituents R⁶, unsubstituted aryloxy or aryloxy substituted by 1 to 3 substituents R⁶, unsubstituted heteroaryloxy or heteroaryloxy substituted by 1 to 3 substituents R⁶ unsubstituted arylC₁-C₆alkyl or heteroarylC₁-C₆alkyl substituted by 1 to 3 substituents R⁶;

and which can be annulated by a saturated, partially unsaturated or aromatic isocyclic or heterocyclic ring to form a bicyclic ring system;

Preferably R³ is an aromatic five-membered heterocycle, and the aromatic five-membered heterocyclic group contains 1 to 4 hetero atoms selected from the group consisting of nitrogen, oxygen and sulfur, it not being possible for each ring system to contain more than 2 oxygen atoms and more than 2 sulfur atoms which and the aromatic five-membered heterocyclic group can be substituted by halogen, cyano, C₁-C₆alkyl, C₁-C₆haloalkyl, C₃-C₆cycloalkyl, C₁-C₆alkoxy, C₁-C₆haloalkoxy, C₂-C₆alkenyl, C₂-C₆alkenyloxy, C₂-C₆haloalkenyloxy, C₂-C₆alkynyloxy, unsubstituted aryl or aryl substituted by 1 to 3 substituents R⁶, unsubstituted heteroaryl or heteroaryl substituted by 1 to 3 substituents R⁶, unsubstituted aryloxy or aryloxy substituted by 1 to 3 substituents R⁶, unsubstituted heteroaryloxy or heteroaryloxy substituted by 1 to 3 substituents R⁶, more preferably R³ is an aromatic five-membered heterocycle, and the aromatic five-membered heterocyclic group contains 1 to 4 hetero atoms selected from the group consisting of nitrogen, oxygen and sulfur, it not being possible for each ring system to contain more than 2 oxygen atoms and more than 2 sulfur atoms which and the aromatic five-membered heterocyclic group can be substituted by halogen, C₁-C₆alkyl, C₁-C₆haloalkyl, C₁-C₆alkoxy, C₁-C₆haloalkoxy, C₂-C₆alkenyloxy, C₂-C₆alkynyloxy, unsubstituted aryl or aryl substituted by 1 to 3 substituents R⁶, unsubstituted aryloxy or aryloxy substituted by 1 to 3 substituents R⁶, and which can be annulated by a saturated, partially unsaturated or aromatic isocyclic or heterocyclic 6 membered ring to form a bicyclic ring system even more preferably R³ is selected from furanyl, benzofuranylthienyl, benzothiophenylthiydiazolyl, pyrrolyl, indolyl, pyrazolyl, indazolyl, imidazolyl, thiazolyl, oxadiazolyl, thiadiazolyl each of which can be substituted by halogen, C₁-C₆alkyl, C₁-C₆haloalkyl or unsubstituted phenyl or phenyl substituted by 1 to 3 substituents selected from Cl and F, most preferably R³ is selected thienyl, pyrazole or thiazolyl each of them can be substituted by halogen, C₁-C₆alkyl, C₁-C₆haloalkyl or unsubstituted phenyl or phenyl substituted by 1 to 3 substituents selected from Cl and F, and which can be annulated by a saturated, partially unsaturated or aromatic isocyclic or heterocyclic 6 membered ring to form a bicyclic ring system. Most preferably R³ is an aromatic five-membered heterocycle, and the aromatic five-membered heterocyclic group contains 1 to 3 hetero atoms selected from the group consisting of nitrogen, oxygen and sulfur, it not being possible for each ring system to contain more than 2 oxygen atoms and more than 2 sulfur atoms which and the aromatic five-membered heterocyclic group can be substituted by halogen, C₁-C₆alkyl, C₁-C₆haloalkyl, C₁-C₆alkoxy, C₁-C₆haloalkoxy, C₂-C₆alkenyloxy, C₂-C₆alkynyloxy, unsubstituted aryl or aryl substituted by 1 to 3 substituents R⁶, unsubstituted aryloxy or aryloxy substituted by 1 to 3 substituents R⁶, and which can be annulated by a saturated, partially unsaturated or aromatic isocyclic or heterocyclic 6 membered ring to form a bicyclic ring system.

R⁵ is hydrogen, C₁-C₆alkyl, C₁-C₆alkoxy or C₃-C₆cycloalkyl.

Preferably R⁵ is hydrogen or C₁-C₆alkyl, more preferably hydrogen or methyl, even more preferably hydrogen.

Preferably R⁶ is hydroxyl, amino, nitro, halogen, C₁-C₆alkyl, C₁-C₆alkoxy, C₁-C₆alkyl, C₁-C₆haloalkyl, C₁-C₆alkoxy or C₁-C₆haloalkoxy; more preferably R⁶ is halogen, C₁-C₆alkyl, C₁-C₆haloalkyl or unsubstituted phenyl or phenyl substituted by 1 to 3 substituents selected from Cl and F.

Preferably the invention relates to compounds of formula (I) wherein

X and Y are both S;
R¹, R² and R⁴ are independently hydrogen, halogen, C₁-C₆alkyl, C₁-C₆haloalkyl, C₁-C₆alkoxy or C₁-C₆haloalkoxy;
R³ is an aromatic five-membered heterocycle, and the aromatic five-membered heterocyclic group contains 1 to 3 hetero atoms selected from the group consisting of nitrogen, oxygen and sulfur, it not being possible for each ring system to contain more than 2 oxygen atoms and more than 2 sulfur atoms which and the aromatic five-membered heterocyclic group can be substituted by halogen, cyano, C₁-C₆alkyl, C₁-C₆haloalkyl, C₃-C₆cycloalkyl, C₁-C₆alkoxy, C₁-C₆haloalkoxy, C₂-C₆alkenyl, C₂-C₆alkenyloxy, C₂-C₆haloalkenyloxy, C₂-C₆alkynyloxy, unsubstituted aryl or aryl substituted by 1 to 3 substituents R⁶, unsubstituted heteroaryl or heteroaryl substituted by 1 to 3 substituents R⁶, unsubstituted aryloxy or aryloxy substituted by 1 to 3 substituents R⁶, unsubstituted heteroaryloxy or heteroaryloxy substituted by 1 to 3 substituents R⁶;
R⁵ is hydrogen, C₁-C₆alkyl, C₁-C₆alkoxy or C₃-C₆cycloalkyl;
R⁶ is independently hydroxyl, amino, nitro, halogen, C₁-C₆alkyl, C₁-C₆alkoxy, C₁-C₆alkyl, C₁-C₆haloalkyl, C₁-C₆alkoxy or C₁-C₆haloalkoxy.

More preferably the invention relates to compounds of formula (I) wherein

X and Y are both S;
R¹, R² and R⁴ are independently hydrogen, C₁-C₆alkyl;
R³ is an aromatic five-membered heterocycle, and the aromatic five-membered heterocyclic group contains 1 to 3 hetero atoms selected from the group consisting of nitrogen, oxygen and sulfur, it not being possible for each ring system to contain more than 2 oxygen atoms and more than 2 sulfur atoms which and the aromatic five-membered heterocyclic group can be substituted by halogen, cyano, C₁-C₆alkyl, C₁-C₆haloalkyl, C₃-C₆cycloalkyl, C₁-C₆alkoxy, C₁-C₆haloalkoxy, C₂-C₆alkenyl, C₂-C₆alkenyloxy, C₂-C₆haloalkenyloxy, C₂-C₆alkynyloxy, unsubstituted aryl or aryl substituted by 1 to 3 substituents R⁶, unsubstituted heteroaryl or heteroaryl substituted by 1 to 3 substituents R⁶, unsubstituted aryloxy or aryloxy substituted by 1 to 3 substituents R⁶, unsubstituted heteroaryloxy or heteroaryloxy substituted by 1 to 3 substituents R⁶;
R⁵ is hydrogen, C₁-C₆alkyl, C₁-C₆alkoxy or C₃-C₆cycloalkyl;
R⁶ is independently hydroxyl, amino, nitro, halogen, C₁-C₆alkyl, C₁-C₆alkoxy, C₁-C₆alkyl, C₁-C₆haloalkyl, C₁-C₆alkoxy or C₁-C₆haloalkoxy;
R⁶ is independently hydroxyl, amino, nitro, halogen, C₁-C₆alkyl, C₁-C₆alkoxy, C₁-C₆alkyl, C₁-C₆haloalkyl, C₁-C₆alkoxy or C₁-C₆haloalkoxy;
R⁶ is independently hydroxyl, amino, nitro, halogen, C₁-C₆alkyl, C₁-C₆alkoxy, C₁-C₆alkyl, C₁-C₆haloalkyl, C₁-C₆alkoxy or C₁-C₆haloalkoxy.

In further preferred compounds of formula (I)

X and Y are independently O or S;
R¹, R² and R⁴ are independently hydrogen, halogen, C₁-C₆alkyl or C₁-C₆haloalkyl;
R³ is an aromatic five-membered heterocycle, and the aromatic five-membered heterocyclic group contains 1 to 3 hetero atoms selected from the group consisting of nitrogen, oxygen and sulfur, it not being possible for each ring system to contain more than 2 oxygen atoms and more than 2 sulfur atoms which and the aromatic five-membered heterocyclic group can be substituted by halogen, C₁-C₆alkyl, C₁-C₆haloalkyl, C₁-C₆alkoxy, C₁-C₆haloalkoxy, C₂-C₆alkenyloxy, C₂-C₆alkynyloxy, unsubstituted aryl or aryl substituted by 1 to 3 substituents R⁶, unsubstituted aryloxy or aryloxy substituted by 1 to 3 substituents R⁶, R⁵ is hydrogen or C₁-C₆alkyl;
R⁶ is halogen, C₁-C₆alkyl, C₁-C₆haloalkyl or unsubstituted phenyl or phenyl substituted by 1 to 3 substituents selected from Cl and F;
R⁶ is independently hydroxyl, amino, nitro, halogen, C₁-C₆alkyl, C₁-C₆alkoxy, C₁-C₆alkyl, C₁-C₆haloalkyl, C₁-C₆alkoxy or C₁-C₆haloalkoxy

In further preferred compounds of formula (I)

X and Y are both S;
R¹, R² and R⁴ are independently hydrogen, halogen, C₁-C₆alkyl or C₁-C₆haloalkyl;
R³ is selected from furanyl, benzofuranylthienyl, benzothiophenylthiydiazolyl, pyrrolyl, indolyl, pyrazolyl, indazolyl, imidazolyl, thiazolyl, oxadiazolyl, thiadiazolyl each of which can be substituted by halogen, C₁-C₆alkyl, C₁-C₆haloalkyl or unsubstituted phenyl or phenyl substituted by 1 to 3 substituents selected from Cl and F;
R⁵ is hydrogen or C₁-C₆alkyl.

More preferably the invention relates to compounds of formula (I) wherein

X and Y are both S;
R¹, R² and R⁴ are independently hydrogen or C₁-C₆alkyl;
R³ is an aromatic five-membered heterocycle, and the aromatic five-membered heterocyclic group contains 1 to 3 hetero atoms selected from the group consisting of nitrogen, oxygen and sulfur, it not being possible for each ring system to contain more than 2 oxygen atoms and more than 2 sulfur atoms which and the aromatic five-membered heterocyclic group can be substituted by halogen, C₁-C₆alkyl, C₁-C₆haloalkyl, C₁-C₆alkoxy, C₁-C₆haloalkoxy, C₂-C₆alkenyloxy, C₂-C₆alkynyloxy, optionally substituted aryl or optionally substituted aryloxy,
R⁵ is hydrogen or methyl.

Even more preferably the invention relates to compounds of formula (I) wherein

X and Y are both S;
R¹, R² and R⁴ are independently hydrogen or methyl;
R³ is selected from furanyl, benzofuranylthienyl, benzothiophenylthiydiazolyl, pyrrolyl, indolyl, pyrazolyl, indazolyl, imidazolyl, thiazolyl, oxadiazolyl, thiadiazolyl each of which can be substituted by halogen, C₁-C₆alkyl, C₁-C₆haloalkyl or unsubstituted phenyl or phenyl substituted by 1 to 3 substituents selected from Cl and F;
R⁵ is hydrogen.

Most preferably the invention relates to compounds of formula (I) wherein

X and Y are both S;
R¹, R² and R⁴ are each hydrogen;
R³ is selected from furanyl, benzofuranylthienyl, benzothiophenylthiydiazolyl, pyrrolyl, indolyl, pyrazolyl, indazolyl, imidazolyl, thiazolyl, oxadiazolyl, thiadiazolyl each of which can be substituted by halogen, C₁-C₆alkyl, C₁-C₆haloalkyl or unsubstituted phenyl or phenyl substituted by 1 to 3 substituents selected from Cl and F;
R⁵ is hydrogen.

The invention also relates to compounds of formula (I-1):

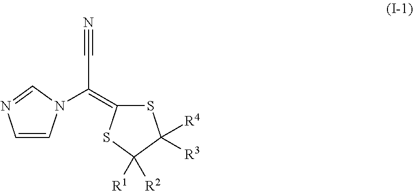

in which R¹, R², R³ and R⁴ have the definitions as described for formula I. Preferred definitions of R¹, R², R³ and R⁴ are as defined for formula I.

The invention also relates to compounds of formula (I-2):

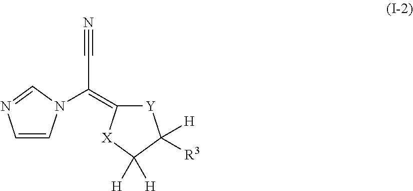

wherein X, Y and R³ have the definition as described for formula I. Preferred definitions of X, Y and R³ are as defined for formula I.

The invention also relates to compounds of formula (I-3):

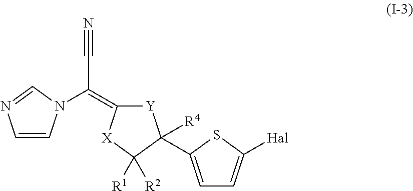

wherein X, Y, R¹, R² and R⁴ have the definition as described for formula (I) and Hal is halogen.

Preferred definitions of X, Y, R¹, R² and R⁴ are as defined for formula I.

In a preferred embodiment the compounds of the present invention are selected from the formula (I.a), (I.b), (I.c), (I.d), (I.e), (I.f), (I.g), (I.h), (I.i), (I.j), (I.k), (I.m), (I.n), (I.o), (I.p), (I.q), (I.r), (I.s), (I.t), (I.u), (I.v), (I.w), (I.x), (I.y), (I.z), (I.aa), (I.ab), (I.ac), (I.ad), (I.ae), (I.af), (I.ag), (I.ah), (I.ai), (I.aj), (I.ak), (I.am), (I.an), (I.ao), (I.ap), (I.aq), (I.ar), (I.as), (I.at), (I.au), (I.av), (I.aw), (I.ax), (I.ay), (I.az), (I.ba), and (I.bb) wherein X and Y are both S and R¹, R² and R⁴ are independently hydrogen or methyl more preferably R¹, R² and R⁴ are each hydrogen.

In a more preferred embodiment the compounds of the present invention are selected from formula (I.e), (I.h), (I.x), (I.v), (I.z), (I.aa), (I.ac), (I.ad), (I.am), (I.am), (I.an), (I.as), and (I.bb) wherein X and Y are both S and R¹, R² and R⁴ are independently hydrogen or methyl more preferably R¹, R² and R⁴ are each hydrogen.

Further preferred embodiments of the present invention are the embodiments E-I.a to E-I.bb, which are defined as compounds of formula (I) which are represented by one formula selected from the group consisting of the formula (I.a) to (I.bb) as described below, wherein in formulae (I.a) to (I.bb) the meanings of the substituents X, Y, R¹, R² and R⁴ have the preferred meanings as mentioned above or one of the meanings 1 to 28 given in the corresponding Table 1.

For example, embodiment E-I.a is represented by the compounds of formula (I.a)

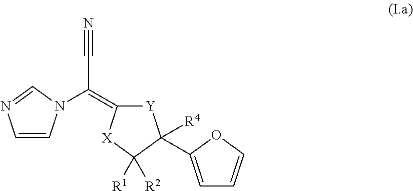

and the substituents X, Y, R¹, R² and R⁴ have the meanings as defined above or one of the meanings 1 to 28 given in the Table 1.

Embodiments E-I.b to E-I.bb are defined accordingly and the substituents X, Y, R¹, R² and R⁴ have the meanings as defined above or one of the meanings 1 to 28 given in the corresponding Table 1.

The invention further relates to a process for the preparation of a compound of formula (I) and to compounds obtainable by this process

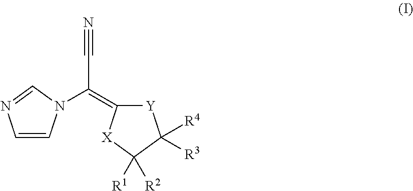

comprising reacting imidazol-1-yl-acetonitrile with compound of formula (III)

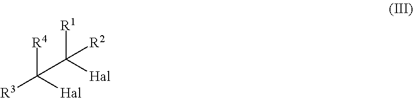

and with a compound of formula (IV)

X═C═Y (IV)

In the presence of a base, wherein X, Y, R¹, R², R³ and R⁴ are as defined as in any of the claims 1 to 7 and Hal is halogen. Preferably the base is sodium hydroxide or potassium hydroxide. Preferably the reaction is carried out at room temperature (=ambient temperature) which denotes a temperature between 10° C. and 30° C., more preferable at 20° C. to 25° C. Preferably the reaction is carried out in a polar aprotic solvent, preferably in dimethylformamide (DMF), acetonitrile (MeCN), or dimethyl sulfoxide (DMSO), most preferably in dimethyl sulfoxide (DMSO).

The invention further relates to a process for the preparation of a compound of formula (I) and to compounds obtainable by this process

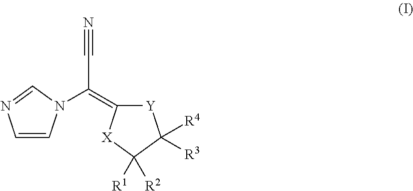

comprising reacting imidazol-1-yl-acetonitrile with compound of formula (XI)

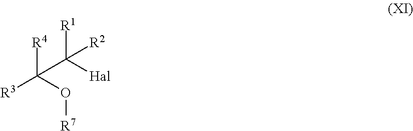

and with a compound of formula (IV)

X═C═Y (IV)

In the presence of a base, wherein X, Y, R¹, R², R³ and R⁴ are as defined as in any of the claims 1 to 7, R⁷ is a sulfonyl group, and Hal is halogen. Preferably the base is sodium hydroxide or potassium hydroxide. Preferably R⁷ is sodium hydroxide or potassium hydroxide. Preferably the reaction is carried out at room temperature (=ambient temperature) which denotes a temperature between 10° C. and 30° C., more preferable at 20° C. to 25° C. Preferably the reaction is carried out in a polar aprotic solvent, preferably in dimethylformamide (DMF), acetonitrile (MeCN), or dimethyl sulfoxide (DMSO), most preferably in dimethyl sulfoxide (DMSO).

Compounds of the present invention can be made as shown in the following schemes, in which, unless otherwise stated, the definition of each variable is as defined above for a compound of formula (I).

The compounds of formula I, wherein X, Y, R¹, R², R³ and R⁴ are as defined for formula I, can be obtained by transformation of imidazol-1-yl-acetonitrile (II), a compound of formula (III), wherein R¹, R², R³ and R⁴ are as defined for formula (I) and Hal is halogen, and a compound of formula (IV), wherein X and Y are as defined for formula I, with a base, such as sodium hydroxide or potassium hydroxide. This is shown in Scheme 1.

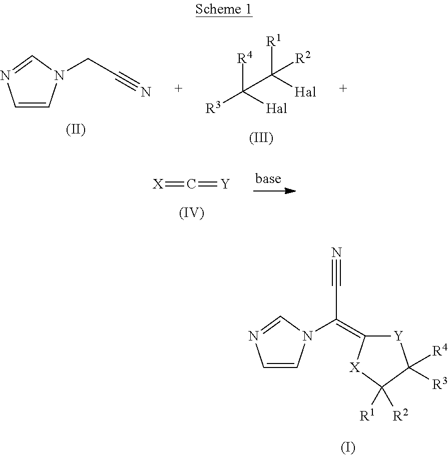

The compounds of formula (III), wherein R¹, R², R³ and R⁴ are as defined for formula (I) and Hal is halogen, can be obtained by transformation of a compound of formula (V), wherein R¹, R², R³ and R⁴ are as defined for formula (I) and Hal is halogen, with a halogenation reagent, such as phosphorus oxychloride, phosphorus oxybromide, thionyl chloride or thionyl bromide. This is shown in Scheme 2.

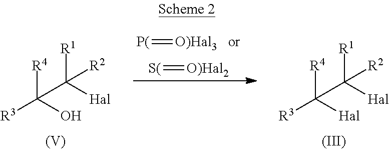

Alternatively, the compounds of formula (III), wherein R¹, R², R³ and R⁴ are as defined for formula (I) and Hal is halogen, can be obtained by transformation of a compound of formula (VI), wherein R¹, R², R³ and R⁴ are as defined for formula I, with a halogenation reagent, such as phosphorus oxychloride, phosphorus oxybromide, thionyl chloride or thionyl bromide. This is shown in Scheme 3.

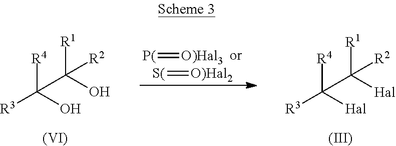

The compounds of formula (V) wherein R¹, R² and R³ are as defined for formula I, R⁴ is hydrogen and Hal is halogen, can be obtained by transformation of a compound of formula (VII), wherein R¹, R² and R³ are as defined for formula (I) and Hal is halogen, with a reducing agent, such as sodium borohydride. This is shown in Scheme 4.

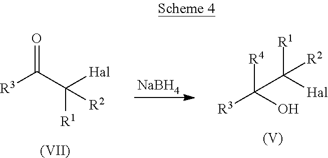

The compounds of formula (VII), wherein R¹, R² and R³ are as defined for formula (I) and Hal is halogen, can be obtained by transformation of a compound of formula (VIII), wherein R¹, R² and R³ are as defined for formula I, with a halogenation reagent, such as chlorine or bromine. This is shown in Scheme 5.

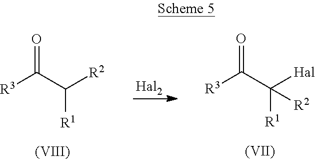

Alternatively, the compounds of formula (V), wherein R¹, R² and R³ are as defined for formula I, R⁴ is hydrogen and Hal is halogen, can be obtained by transformation of a compound of formula (IX), wherein R³ is as defined for formula I, with a compound of formula (X), wherein R¹ and R² are as defined for formula (I) and Hal is halogen, preferably chloro or bromo, and a base. This is shown in Scheme 6.

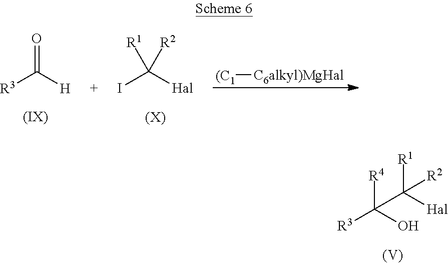

Alternatively, the compounds of formula I, wherein X, Y, R¹, R², R³ and R⁴ are as defined for formula I, can be obtained by transformation of imidazol-1-yl-acetonitrile (II), a compound of formula (XI), wherein R¹, R², R³ and R⁴ are as defined for formula I, R⁶ is a sulfonyl group, such as methylsulfonyl or p-tolylsulfonyl, and Hal is halogen, and a compound of formula (IV), wherein X and Y are as defined for formula I, with a base, such as sodium hydroxide or potassium hydroxide. This is shown in Scheme 7.

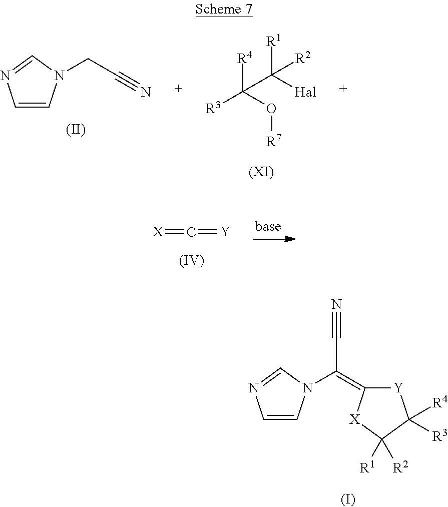

The compounds of formula (XI), wherein R¹, R², R³ and R⁴ are as defined for formula I, R⁷ is a sulfonyl group, such as methylsulfonyl or p-tosylsulfonyl, and Hal is halogen, can be obtained by transformation of a compound of formula (V), wherein R¹, R², R³ and R⁴ are as defined for formula (I) and Hal is halogen, with a sulfonyl halide, such as mesyl chloride or tosyl chloride. This is shown in Scheme 8.

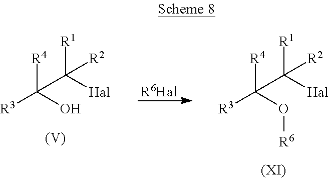

The compounds of formula (I) of this invention are useful as plant disease control agents. The present invention therefore further comprises a method for controlling plant diseases caused by fungal plant pathogens comprising applying to the plant or portion thereof to be protected, or to the plant seed to be protected, an effective amount of a compound of the invention or a fungicidal composition containing said compound.

Compounds of formula (I) and fungicidal compositions containing them may be used to control plant diseases caused by a broad spectrum of fungal plant pathogens in the Basidiomycete, Ascomycete, Oomycete and/or Deuteromycete, Blasocladiomycete, Chrytidiomycete, Glomeromycete and/or Mucoromycete classes.

They are effective in controlling a broad spectrum of plant diseases, such as foliar pathogens of ornamental, turf, vegetable, field, cereal, and fruit crops.

These pathogens may include:

Oomycetes, including Phytophthora diseases such as those caused by Phytophthora capsici, Phytophthora infestans, Phytophthora sojae, Phytophthora fragariae, Phytophthora nicotianae, Phytophthora cinnamomi, Phytophthora citricola, Phytophthora citrophthora and Phytophthora erythroseptica; Pythium diseases such as those caused by Pythium aphanidermatum, Pythium arrhenomanes, Pythium graminicola, Pythium irregulare and Pythium ultimum; diseases caused by Peronosporales such as Peronospora destructor, Peronospora parasitica, Plasmopara viticola, Plasmopara halstedii, Pseudoperonospora cubensis, Albugo candida, Sclerophthora macrospora and Bremia lactucae; and others such as Aphanomyces cochlioides, Labyrinthula zosterae, Peronosclerospora sorghi and Sclerospora graminicola.

Ascomycetes, including blotch, spot, blast or blight diseases and/or rots for example those caused by Pleosporales such as Stemphylium solani, Stagonospora tainanensis, Spilocaea oleaginea, Setosphaeria turcica, Pyrenochaeta lycoperisici, Pleospora herbarum, Phoma destructiva, Phaeosphaeria herpotrichoides, Phaeocryptocus gaeumannii, Ophiosphaerella graminicola, Ophiobolus graminis, Leptosphaeria maculans, Hendersonia creberrima, Helminthosporium triticirepentis, Setosphaeria turcica, Drechslera glycines, Didymella bryoniae, Cycloconium oleagineum, Corynespora cassiicola, Cochliobolus sativus, Bipolaris cactivora, Venturia inaequalis, Pyrenophora teres, Pyrenophora tritici-repentis, Alternaria alternata, Alternaria brassicicola, Alternaria solani and Alternaria tomatophila, Capnodiales such as Septoria tritici, Septoria nodorum, Septoria glycines, Cercospora arachidicola, Cercospora sojina, Cercospora zeae-maydis, Cercosporella capsellae and Cercosporella herpotrichoides, Cladosporium carpophilum, Cladosporium effusum, Passalora fulva, Cladosporium oxysporum, Dothistroma septosporum, Isariopsis clavispora, Mycosphaerella fijiensis, Mycosphaerella graminicola, Mycovellosiella koepkeii, Phaeoisariopsis bataticola, Pseudocercospora vitis, Pseudocercosporella herpotrichoides, Ramularia beticola, Ramularia collo-cygni, Magnaporthales such as Gaeumannomyces graminis, Magnaporthe grisea, Pyricularia oryzae, Diaporthales such as Anisogramma anomala, Apiognomonia errabunda, Cytospora platani, Diaporthe phaseolorum, Discula destructiva, Gnomonia fructicola, Greeneria uvicola, Melanconium juglandinum, Phomopsis viticola, Sirococcus clavigignenti-juglandacearum, Tubakia dryina, Dicarpella spp., Valsa ceratosperma, and others such as Actinothyrium graminis, Ascochyta pisi, Aspergillus flavus, Aspergillus fumigatus, Aspergillus nidulans, Asperisporium caricae, Blumeriella jaapii, Candida spp., Capnodium ramosum, Cephaloascus spp., Cephalosporium gramineum, Ceratocystis paradoxa, Chaetomium spp., Hymenoscyphus pseudoalbidus, Coccidioides spp., Cylindrosporium padi, Diplocarpon malae, Drepanopeziza campestris, Elsinoe ampelina, Epicoccum nigrum, Epidermophyton spp., Eutypa lata, Geotrichum candidum, Gibellina cerealis, Gloeocercospora sorghi, Gloeodes pomigena, Gloeosporium perennans; Gloeotinia temulenta, Griphospaeria corticola, Kabatiella lini, Leptographium microsporum, Leptosphaerulinia crassiasca, Lophodermium seditiosum, Marssonina graminicola, Microdochium nivale, Monilinia fructicola, Monographella albescens, Monosporascus cannonballus, Naemacyclus spp., Ophiostoma novo-ulmi, Paracoccidioides brasiliensis, Penicillium expansum, Pestalotia rhododendri, Petriellidium spp., Pezicula spp., Phialophora gregata, Phyllachora pomigena, Phymatotrichum omnivora, Physalospora abdita, Plectosporium tabacinum, Polyscytalum pustulans, Pseudopeziza medicaginis, Pyrenopeziza brassicae, Ramulispora sorghi, Rhabdocline pseudotsugae, Rhynchosporium secalis, Sacrocladium oryzae, Scedosporium spp., Schizothyrium pomi, Sclerotinia sclerotiorum, Sclerotinia minor; Sclerotium spp., Typhula ishikariensis, Seimatosporium mariae, Lepteutypa cupressi, Septocyta ruborum, Sphaceloma perseae, Sporonema phacidioides, Stigmina palmivora, Tapesia yallundae, Taphrina bullata, Thielviopsis basicola, Trichoseptoria fructigena, Zygophiala jamaicensis; powdery mildew diseases for example those caused by Erysiphales such as Blumeria graminis, Erysiphe polygoni, Uncinula necator, Sphaerotheca fuligena, Podosphaera leucotricha, Podospaera macularis Golovinomyces cichoracearum, Leveillula taurica, Microsphaera diffusa, Oidiopsis gossypii, Phyllactinia guttata and Oidium arachidis; molds for example those caused by Botryosphaeriales such as Dothiorella aromatica, Diplodia seriata, Guignardia bidwellii, Botrytis cinerea, Botryotinia allii, Botryotinia fabae, Fusicoccum amygdali, Lasiodiplodia theobromae, Macrophoma theicola, Macrophomina phaseolina, Phyllosticta cucurbitacearum; anthracnoses for example those caused by Glommerelales such as Colletotrichum gloeosporioides, Colletotrichum lagenarium, Colletotrichum gossypii, Glomerella cingulata, and Colletotrichum graminicola; and wilts or blights for example those caused by Hypocreales such as Acremonium strictum, Claviceps purpurea, Fusarium culmorum, Fusarium graminearum, Fusarium virguliforme, Fusarium oxysporum, Fusarium subglutinans, Fusarium oxysporum f.sp. cubense, Gerlachia nivale, Gibberella fujikuroi, Gibberella zeae, Gliocladium spp., Myrothecium verrucaria, Nectria ramulariae, Trichoderma viride, Trichothecium roseum, and Verticillium theobromae.

Basidiomycetes, including smuts for example those caused by Ustilaginales such as Ustilaginoidea virens, Ustilago nuda, Ustilago tritici, Ustilago zeae, rusts for example those caused by Pucciniales such as Cerotelium fici, Chrysomyxa arctostaphyli, Coleosporium ipomoeae, Hemileia vastatrix, Puccinia arachidis, Puccinia cacabata, Puccinia graminis, Puccinia recondita, Puccinia sorghi, Puccinia hordei, Puccinia striiformis f.sp. Hordei, Puccinia striiformis f.sp. Secalis, Pucciniastrum coryli, or Uredinales such as Cronartium ribicola, Gymnosporangium juniperi-viginianae, Melampsora medusae, Phakopsora pachyrhizi, Phragmidium mucronatum, Physopella ampelosidis, Tranzschelia discolor and Uromyces viciae-fabae; and other rots and diseases such as those caused by Cryptococcus spp., Exobasidium vexans, Marasmiellus inoderma, Mycena spp., Sphacelotheca reiliana, Typhula ishikariensis, Urocystis agropyri, Itersonilia perplexans, Corticium invisum, Laetisaria fuciformis, Waitea circinata, Rhizoctonia solani, Thanetephorus cucurmeris, Entyloma dahliae, Entylomella microspora, Neovossia moliniae and Tilletia caries.

Blastocladiomycetes, such as Physoderma maydis.

Mucoromycetes, such as Choanephora cucurbitarum.; Mucor spp.; Rhizopus arrhizus,

As well as diseases caused by other species and genera closely related to those listed above.

In addition to their fungicidal activity, the compounds and compositions comprising them may also have activity against bacteria such as Erwinia amylovora, Erwinia caratovora, Xanthomonas campestris, Pseudomonas syringae, Strptomyces scabies and other related species as well as certain protozoa.

Compounds of formula (I) may be mixed with one or more of compounds selected from those in the following chemical or functional classes:— 1,2,4-thiadiazoles, 2,6-dinitroanilines, acylalanines, aliphatic nitrogenous compounds, amidines, aminopyrimidinols, anilides, anilino-pyrimidines, anthraquinones, antibiotics, aryl-phenylketones, benzamides, benzene-sulfonamides, benzimidazoles, benzothiazoles, benzothiodiazoles, benzothiophenes, benzoylpyridines, benzthiadiazoles, benzylcarbamates, butylamines, carbamates, carboxamides, carpropamids, chloronitriles, cinnamic acid amides, copper containing compounds, cyanoacetamideoximes, cyanoacrylates, cyanoimidazoles, cyanomethylene-thiazolidines, dicarbonitriles, dicarboxamides, dicarboximides, dimethylsulphamates, dinitrophenol carbonates, dinitrophenysl, dinitrophenyl crotonates, diphenyl phosphates, dithiino compounds, dithiocarbamates, dithioethers, dithiolanes, ethyl-amino-thiazole carboxamides, ethyl-phosphonates, furan carboxamides, glucopyranosyls, glucopyranoxyls, glutaronitriles, guanidines, herbicides/plant growth regulatosr, hexopyranosyl antibiotics, hydroxy(2-amino)pyrimidines, hydroxyanilides, hydroxyisoxazoles, imidazoles, imidazolinones, insecticides/plant growth regulators, isobenzofuranones, isoxazolidinyl-pyridines, isoxazolines, maleimides, mandelic acid amides, mectin derivatives, morpholines, norpholines, n-phenyl carbamates, organotin compounds, oxathiin carboxamides, oxazoles, oxazolidine-diones, phenols, phenoxy quinolines, phenyl-acetamides, phenylamides, phenylbenzamides, phenyl-oxo-ethyl-thiophenes amides, phenylpyrroles, phenylureas, phosphorothiolates, phosphorus acids, phthalamic acids, phthalimides, picolinamides, piperazines, piperidines, plant extracts, polyoxins, propionamides, pthalimides, pyrazole-4-carboxamides, pyrazolinones, pyridazinones, pyridines, pyridine carboxamides, pyridinyl-ethyl benzamides, pyrimdinamines, pyrimidines, pyrimidine-amines, pyrimidione-hydrazone, pyrrolidines, pyrrolquinoliones, quinazolinones, quinolines, quinoline derivatives, quinoline-7-carboxylic acids, quinoxalines, spiroketalamines, strobilurins, sulfamoyl triazoles, sulphamides, tetrazolyloximes, thiadiazines, thiadiazole carboxamides, thiazole carboxanides, thiocyanates, thiophene carboxamides, toluamides, triazines, triazobenthiazoles, triazoles, triazole-thiones, triazolo-pyrimidylamine, valinamide carbamates, ammonium methyl phosphonates, arsenic-containing compounds, benyimidazolylcarbamates, carbonitriles, carboxanilides, carboximidamides, carboxylic phenylamides, diphenyl pyridines, furanilides, hydrazine carboxamides, imidazoline acetates, isophthalates, isoxazolones, mercury salts, organomercury compounds, organophosphates, oxazolidinediones, pentylsulfonyl benzenes, phenyl benzamides, phosphonothionates, phosphorothioates, pyridyl carboxamides, pyridyl furfuryl ethers, pyridyl methyl ethers, SDHIs, thiadiazinanethiones, thiazolidines.

Particularly preferred fungicidal combinations include the following where “I” designates compounds of formula (I): I+(.+/−.)-cis-1-(4-chlorophenyl)-2-(1H-1,2,4-triazol-1-yl)-cycloheptanol (huanjunzuo), I+(2RS)-2-bromo-2-(bromomethyl)glutaronitrile (bromothalonil), I+(E)-N-methyl-2-[2-(2, 5-dimethylphenoxymethyl) phenyl]-2-methoxy-iminoacetamide, (mandestrobin), I+1-(5-bromo-2-pyridyl)-2-(2,4-difluorophenyl)-1,1-difluoro-3-(1,2,4-triazol-1-yl)propan-2-ol, I+1-methylcyclopropene, I+2-methyl-, [[4-methoxy-2-[[[(3S,7R,8R,9S)-9-methyl-8-(2-methyl-1-oxopropoxy)-2,6-dioxo-7-(phenylmethyl)-1,5-dioxonan-3-yl]amino]carbonyl]-3-pyridinyl]oxy]propanoic acid methyl ester, I+2-(1-tert-butyl)-1-(2-chlorophenyl)-3-(1,2,4-triazol-1-yl)-propan-2-ol (TCDP), I+2,4-D, I+2,4-DB, I+2,6-dichloro-N-(4-trifluoromethylbenzyl)-benzamide, I+2,6-dimethyl-[1,4]dithiino[2,3-c:5,6-c′]dipyrrole-1,3,5,7(2H,6H)-tetraone, I+2-[[(1R,5S)-5-[(4-fluorophenyl)methyl]-1-hydroxy-2,2-dimethyl-cyclopentyl]methyl]-4H-1,2,4-triazole-3-thione I+2-[[3-(2-chlorophenyl)-2-(2,4-difluorophenyl)oxiran-2-yl]methyl]-4H-1,2,4-triazole-3-thione I+ametoctradin (imidium), I+2-[2-[(7,8-difluoro-2-methyl-3-quinolyl)oxy]-6-fluoro-phenyl]propan-2-ol I+2-[2-fluoro-6-[(8-fluoro-2-methyl-3-quinolyl)oxy]phenyl]propan-2-ol I+cyflufenamid, I+2-benzyl-4-chlorophenol (Chlorophene), I+3-(difluoromethyl)-N-(7-fluoro-1,1,3,3-tetramethyl-indan-4-yl)-1-methyl-pyrazole-4-carboxamide I+diclocymet, I+3-(difluoromethyl)-N-methoxy-1-methyl-N-[1-methyl-2-(2,4,6-trichlorophenyl)ethyl]pyrazole-4-carboxamide, I+3′-chloro-2-methoxy-N-[(3RS)-tetrahydro-2-oxofuran-3-yl]acet-2′,6′-xylidide (clozylacon), I+3-iodo-2-propinyl n-butylcarbamate (IPBC), I+4,4,5-trifluoro-3,3-dimethyl-1-(3-quinolyl)isoquinoline I+4,4-difluoro-3,3-dimethyl-1-(3-quinolyl)isoquinoline I+5-fluoro-3,3,4,4-tetramethyl-1-(3-quinolyl)isoquinoline I+9-fluoro-2,2-dimethyl-5-(3-quinolyl)-3H-1,4-benzoxazepine I+tebufloquin, I+4-CPA, I+5-fluoro-2-(p-tolylmethoxy)pyrimidin-4-amine I+ferimzone, I+acibenzolar, I+acibenzolar-S-methyl, I+allyl alcohol, I+ametoctradin, I+amisulbrom, I+anilazine, I+aureofungin, I+azaconazole, I+azafenidin, I+azithiram, I+azoxystrobin, I+benalaxyl, I+benalaxyl-M, I+benalaxyl-M (kiralaxyl), I+benomyl, I+benthiavalicarb, I+benthiazole (TCMTB), I+benzalkonium chloride, I+benzamorf, I+benzovindiflupyr (solatenol), I+bethoxazin, I+biphenyl, I+bitertanol (biloxazol), I+bixafen, I+BLAD, I+blasticidin-S, I+Bordeaux mixture, I+boscalid, I+bromuconazole, I+bupirimate, I+but-3-ynyl N-[6-[[(Z)-[(1-methyltetrazol-5-yl)-phenyl-methylene]amino]oxymethyl]-2-pyridyl]carbamate I+dazomet, I+butylamine, I+calcium polysulfide, I+captafol, I+captan, I+carbaryl, I+carbendazim, I+carbendazim chlorhydrate, I+carboxin, I+CAS 517875-34-2 (DAS777), I+chinomethionate, I+chinomethionate (oxythioquinox, quinoxymethionate), I+chitosan, I+chlobenthiazone, I+chlorfenazole, I+chlormequat, I+chloroneb, I+chloropicrin, I+chlorothalonil, I+chlozolinate, I+climbazole, I+clofencet, I+copper acetate, I+copper carbonate, I+copper hydroxide, I+copper naphthenate, I+copper oleate, I+copper oxychloride, I+copper oxyquinolate, I+copper silicate, I+copper sulphate, I+copper tallate, I+coumoxystrobin, I+cresol, I+cuprous oxide, I+cyazofamid, I+cyclafuramid, I+cymoxanil, I+cyproconazole, I+cyprodinil, I+daracide, I+dichlofluanid, I+dichlorophen (dichlorophene), I+dichlorprop, I+diclomezine, I+dicloran, I+diethofencarb, I+difenoconazole, I+difenzoquat, I+diflumetorim, I+dimetachlone (dimethaclone), I+dimetconazole, I+dimethipin, I+dimethirimol, I+dimethomorph, I+dimoxystrobin, I+dingjunezuo (Jun Si Qi), I+diniconazole, I+diniconazole-M, I+, I+dinobuton, I+dinocap, I+dinocton, I+dinopenton, I+diphenylamine, I+dipyrithione, I+ditalimfos, I+dithianon, I+dithioether, I+dodemorph, I+dodicin, I+dodine, I+doguadine, I+drazoxolon, I+edifenphos, I+endothal, I+enestroburin, enoxastrobin I+fenamistrobin, I+epoxiconazole, I+etaconazole, I+etem, I+ethaboxam, I+ethephon, I+ethoxyquin, I+famoxadone, I+fenamidone, I+fenarimol, I+fenbuconazole, I+fenfuram, I+fenhexamid, I+fenoxanil, I+fenpiclonil, I+fenpropidin, I+fenpropimorph, I+fenpyrazamine, I+fentin acetate, I+fentin hydroxide, I+ferbam, I+fluazinam, I+fludioxonil, I+flufenoxystrobin, I+flumetralin, I+flumorph, I+fluopicolide, I+fluopicolide (flupicolide), I+fluopyram, I+fluoroimide, I+fluoxastrobin, I+fluquinconazole, I+flusilazole, I+flusulfamide, I+flutianil, I+flutolanil, I+flutriafol, I+fluxapyroxad, I+folpet, I+forchlorfenuron, I+fosetyl, I+fuberidazole, I+furalaxyl, I+furametpyr, I+gibberellic acid, I+gibberellins, I+guazatine, I+hexachlorobenzene, I+hexaconazole, I+hymexazol, I+hymexazole, hydroxyisoxazole I+imazalil, I+I+etridiazole, I+imazalil, I+imazalil sulphate, I+imibenconazole, I+iminoctadine, I+iminoctadine triacetate, I+iodocarb (isopropanyl butylcarbamate), I+ipconazole, I+iprobenfos, I+iprodione, I+iprovalicarb, I+isofetamid, I+isopropanyl butylcarbamate (iodocarb), I+isoprothiolane, I+isopyrazam, I+isotianil, I+kasugamycin, I+kresoxim-methyl, I+KSF-1002, I+maleic hydrazide, I+mancozeb, I+mandestrobin, I+mandipropamid, I+maneb, I+mepanipyrim, I+mepiquat, I+mepronil, I+meptyldinocap, I+metalaxyl, I+metalaxyl-M (mefenoxam), I+metam, I+metaminostrobin, I+metconazole, I+methyl bromide, I+methyl iodide, I+methyl isothiocyanate, I+metiram (polyram), I+metiram-zinc, I+metominostrobin, I+metrafenone, I+m-phenylphenol, I+myclobutanil, I+N′-(2,5-Dimethyl-4-phenoxy-phenyl)-N-ethyl-N-methyl-formamidine, I+N′-[4-(4,5-Dichloro-thiazol-2-yloxy)-2,5-dimethyl-phenyl]-N-ethyl-N-methyl-formamidine, I+N′-[4-[[3-[(4-chlorophenyl)methyl]-1,2,4-thiadiazol-5-yl]oxy]-2,5-dimethyl-phenyl]-N-ethyl-N-methyl-formamidine, I+ethirimol, I+N-(2-p-chlorobenzoylethyl)-hexaminium chloride, I+N-[(5-chloro-2-isopropyl-phenyl)methyl]-N-cyclopropyl-3-(difluoromethyl)-5-fluoro-1-methyl-pyrazole-4-carboxamide I+N-cyclopropyl-3-(difluoromethyl)-5-fluoro-N-[(2-isopropylphenyl)methyl]-1-methyl-pyrazole-4-carboxamide I+carpropamid, I+nabam, I+naphthalene acetamide, I+NNF-0721, I+octhilinone, I+ofurace, I+orysastrobin, I+osthol, I+oxadixyl, I+oxasulfuron, I+oxathiapiprolin, I+oxine-copper, I+oxolinic acid, I+oxpoconazole, I+oxycarboxin, I+paclobutrazol, I+pefurazoate, I+penconazole, I+pencycuron, I+penflufen, I+penthiopyrad, I+phenamacril, I+phosdiphen, I+phosetyl-Al, I+phosetyl-Al (fosetyl-al), I+phosphorus acids, I+phthalide (fthalide), I+picarbutrazox, I+picoxystrobin, I+piperalin, I+polycarbamate, I+polyoxin D (polyoxrim), I+p-phenylphenol, I+probenazole, I+prochloraz, I+procymidone, I+prohexadione, I+prohexadione-calcium, I+propamidine, I+propamocarb, I+propiconazole, I+propineb, I+propionic acid, I+proquinazid, I+prothioconazole, I+pyraclostrobin, I+pyrametostrobin, I+pyraoxystrobin, I+pyrazophos, I+pyribencarb (KIF-7767), I+pyrifenox, I+pyrimethanil, I+pyriofenone (IKF-309), I+pyroquilon, I+quinoxyfen, I+quintozene, I+sedaxane, I+silthiofam, I+simeconazole, I+spiroxamine, I+streptomycin, I+sulphur, I+tebuconazole, I+tebufloquin, I+tecloftalam, I+tecnazene, (TCNB), I+tetraconazole, I+thiabendazole, I+thicyofen, I+thidiazuron, I+thifluzamide, I+thiophanate-methyl, I+thiram, I+tiadinil, I+tioxymid, I+tolclofos-methyl, I+tolprocarb, I+tolylfluanid, I+triadimefon, I+triadimenol, I+triazoxide, I+tribromophenol (TBP), I+tribufos (tributyl phosphorotrithioate), I+triclopyricarb, I+tricyclazole, I+tridemorph, I+trifloxystrobin, I+triflumizole, I+triforine, I+trinexapac, I+triticonazole, I+uniconazole, I+validamycin, I+valifenalate, I+vapam, I+vapam (metam sodium), I+vinclozolin, I+zineb, I+ziram, I+zoxamide, I+α-naphthalene acetic acid.

Compounds of this invention can also be mixed with one or more further pesticides including insecticides, nematocides, bactericides, acaricides, growth regulators, chemosterilants, semiochemicals, repellents, attractants, pheromones, feeding stimulants or other biologically active compounds to form a multi-component pesticide giving an even broader spectrum of agricultural protection.

Examples of such agricultural protectants with which compounds of this invention can be formulated are:

Insecticides such as abamectin, acephate, acetamiprid, amidoflumet (S-1955), avermectin, azadirachtin, azinphos-methyl, bifenthrin, bifenazate, buprofezin, carbofuran, cartap, chlorantraniliprole (DPX-E2Y45), chlorfenapyr, chlorfluazuron, chlorpyrifos, chlorpyrifos-methyl, chromafenozide, clothianidin, cyflumetofen, cyfluthrin, beta-cyfluthrin, cyhalothrin, lambda-cyhalothrin, cypermethrin, cyromazine, deltamethrin, diafenthiuron, diazinon, dieldrin, diflubenzuron, dimefluthrin, dimethoate, dinotefuran, diofenolan, emamectin, endosulfan, esfenvalerate, ethiprole, fenothiocarb, fenoxycarb, fenpropathrin, fenvalerate, fipronil, flonicamid, flubendiamide, flucythrinate, tau-fluvalinate, flufenerim (UR-50701), flufenoxuron, fonophos, halofenozide, hexaflumuron, hydramethylnon, imidacloprid, indoxacarb, isofenphos, lufenuron, malathion, metaflumizone, metaldehyde, methamidophos, methidathion, methomyl, methoprene, methoxychlor, metofluthrin, monocrotophos, methoxyfenozide, nitenpyram, nithiazine, novaluron, noviflumuron (XDE-007), oxamyl, parathion, parathion-methyl, permethrin, phorate, phosalone, phosmet, phosphamidon, pirimicarb, profenofos, profluthrin, pymetrozine, pyrafluprole, pyrethrin, pyridalyl, pyrifluquinazon, pyriprole, pyriproxyfen, rotenone, ryanodine, spinetoram, spinosad, spirodiclofen, spiromesifen (BSN 2060), spirotetramat, sulprofos, tebufenozide, teflubenzuron, tefluthrin, terbufos, tetrachlorvinphos, thiacloprid, thiamethoxam, thiodicarb, thiosultap-sodium, tralomethrin, triazamate, trichlorfon and triflumuron;

Bactericides such as streptomycin;

Acaricides such as amitraz, chinomethionat, chlorobenzilate, cyenopyrafen, cyhexatin, dicofol, dienochlor, etoxazole, fenazaquin, fenbutatin oxide, fenpropathrin, fenpyroximate, hexythiazox, propargite, pyridaben and tebufenpyrad; and

Biological agents such as Bacillus thuringiensis, Bacillus thuringiensis delta endotoxin, baculovirus, and entomopathogenic bacteria, virus and fungi.

Plant disease control is ordinarily accomplished by applying an effective amount of a compound of this invention either pre- or post-infection, to the portion of the plant to be protected such as the roots, stems, foliage, fruit, seeds, tubers or bulbs, or to the media (soil or sand) in which the plants to be protected are growing. The compounds may also be applied to seeds to protect the seeds and seedlings developing from the seeds. The compounds may also be applied through irrigation water to treat plants.

The present invention envisages application of the compounds of the invention to plant propagation material prior to, during, or after planting, or any combination of these.

Although active ingredients can be applied to plant propagation material in any physiological state, a common approach is to use seeds in a sufficiently durable state to incur no damage during the treatment process. Typically, seed would have been harvested from the field; removed from the plant; and separated from any cob, stalk, outer husk, and surrounding pulp or other non-seed plant material. Seed would preferably also be biologically stable to the extent that treatment would not cause biological damage to the seed. It is believed that treatment can be applied to seed at any time between seed harvest and sowing of seed including during the sowing process.

Methods for applying or treating active ingredients on to plant propagation material or to the locus of planting are known in the art and include dressing, coating, pelleting and soaking as well as nursery tray application, in furrow application, soil drenching, soil injection, drip irrigation, application through sprinklers or central pivot, or incorporation into soil (broad cast or in band). Alternatively or in addition active ingredients may be applied on a suitable substrate sown together with the plant propagation material.

Rates of application for these compounds can be influenced by many factors of the environment and should be determined under actual use conditions. Foliage can normally be protected when treated at a rate of from less than about 1 g/ha to about 5,000 g/ha of active ingredient. Seed and seedlings can normally be protected when seed is treated at a rate of from about 0.1 to about 10 g per kilogram of seed.

Crops of useful plants in which the composition according to the invention can be used include perennial and annual crops, such as berry plants for example blackberries, blueberries, cranberries, raspberries and strawberries; cereals for example barley, maize (corn), millet, oats, rice, rye, sorghum triticale and wheat; fibre plants for example cotton, flax, hemp, jute and sisal; field crops for example sugar and fodder beet, coffee, hops, mustard, oilseed rape (canola), poppy, sugar cane, sunflower, tea and tobacco; fruit trees for example apple, apricot, avocado, banana, cherry, citrus, nectarine, peach, pear and plum; grasses for example Bermuda grass, bluegrass, bentgrass, centipede grass, fescue, ryegrass, St. Augustine grass and Zoysia grass; herbs such as basil, borage, chives, coriander, lavender, lovage, mint, oregano, parsley, rosemary, sage and thyme; legumes for example beans, lentils, peas and soya beans; nuts for example almond, cashew, ground nut, hazelnut, peanut, pecan, pistachio and walnut; palms for example oil palm; ornamentals for example flowers, shrubs and trees; other trees, for example cacao, coconut, olive and rubber; vegetables for example asparagus, aubergine, broccoli, cabbage, carrot, cucumber, garlic, lettuce, marrow, melon, okra, onion, pepper, potato, pumpkin, rhubarb, spinach and tomato; and vines for example grapes.

Crops are to be understood as being those which are naturally occurring, obtained by conventional methods of breeding, or obtained by genetic engineering. They include crops which contain so-called output traits (e.g. improved storage stability, higher nutritional value and improved flavour).

Crops are to be understood as also including those crops which have been rendered tolerant to herbicides like bromoxynil or classes of herbicides such as ALS-, EPSPS-, GS-, HPPD- and PPO-inhibitors. An example of a crop that has been rendered tolerant to imidazolinones, e.g. imazamox, by conventional methods of breeding is Clearfield® summer canola. Examples of crops that have been rendered tolerant to herbicides by genetic engineering methods include e.g. glyphosate- and glufosinate-resistant maize varieties commercially available under the trade names RoundupReady®, Herculex I® and LibertyLink®.

Crops are also to be understood as being those which naturally are or have been rendered resistant to harmful insects. This includes plants transformed by the use of recombinant DNA techniques, for example, to be capable of synthesising one or more selectively acting toxins, such as are known, for example, from toxin-producing bacteria. Examples of toxins which can be expressed include 8-endotoxins, vegetative insecticidal proteins (Vip), insecticidal proteins of bacteria colonising nematodes, and toxins produced by scorpions, arachnids, wasps and fungi.

An example of a crop that has been modified to express the Bacillus thuringiensis toxin is the Bt maize KnockOut® (Syngenta Seeds). An example of a crop comprising more than one gene that codes for insecticidal resistance and thus expresses more than one toxin is VipCot® (Syngenta Seeds). Crops or seed material thereof can also be resistant to multiple types of pests (so-called stacked transgenic events when created by genetic modification). For example, a plant can have the ability to express an insecticidal protein while at the same time being herbicide tolerant, for example Herculex I® (Dow AgroSciences, Pioneer Hi-Bred International).

The compounds according to the invention can be used as pesticidal agents in unmodified form, but they are generally formulated into compositions in various ways using formulation adjuvants, such as carriers, solvents and surface-active substances. The formulations can be in various physical forms, e.g. in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO and WHO Specifications for Pesticides, United Nations, First Edition, Second Revision (2010). Such formulations can either be used directly or diluted prior to use.

The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof.

The active ingredients can also be contained in very fine microcapsules. Microcapsules contain the active ingredients in a porous carrier. This enables the active ingredients to be released into the environment in controlled amounts (e.g. slow-release). Microcapsules usually have a diameter of from 0.1 to 500 microns. They contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes can comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other polymers that are known to the person skilled in the art. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of the compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxypropanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol, propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydrofurfuryl alcohol, hexanol, octanol, ethylene glycol, propylene glycol, glycerol, N-methyl-2-pyrrolidone and the like.

Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances.

A large number of surface-active substances can advantageously be used in both solid and liquid formulations, especially in those formulations which can be diluted with a carrier prior to use.

Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecylbenzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; and also further substances described e.g. in McCutcheon's Detergents and Emulsifiers Annual, MC Publishing Corp., Ridgewood N.J. (1981).

Further adjuvants that can be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and liquid and solid fertilisers.

The compositions according to the invention can include an additive comprising an oil of vegetable or animal origin, a mineral oil, alkyl esters of such oils or mixtures of such oils and oil derivatives. The amount of oil additive in the composition according to the invention is generally from 0.01 to 10%, based on the mixture to be applied. For example, the oil additive can be added to a spray tank in the desired concentration after a spray mixture has been prepared. Preferred oil additives comprise mineral oils or an oil of vegetable origin, for example rapeseed oil, olive oil or sunflower oil, emulsified vegetable oil, alkyl esters of oils of vegetable origin, for example the methyl derivatives, or an oil of animal origin, such as fish oil or beef tallow. Preferred oil additives comprise alkyl esters of C₈-C₂₂ fatty acids, especially the methyl derivatives of C₁₂-C₁₈ fatty acids, for example the methyl esters of lauric acid, palmitic acid and oleic acid (methyl laurate, methyl palmitate and methyl oleate, respectively). Many oil derivatives are known from the Compendium of Herbicide Adjuvants, 10^{th} Edition, Southern Illinois University, 2010.

The inventive compositions generally comprise from 0.1 to 99% by weight, especially from 0.1 to 95% by weight, of compounds of the present invention and from 1 to 99.9% by weight of a formulation adjuvant which preferably includes from 0 to 25% by weight of a surface-active substance. Whereas commercial products may preferably be formulated as concentrates, the end user will normally employ dilute formulations.

The rates of application vary within wide limits and depend on the nature of the soil, the method of application, the crop plant, the pest to be controlled, the prevailing climatic conditions, and other factors governed by the method of application, the time of application and the target crop. As a general guideline compounds may be applied at a rate of from 1 to 2000 l/ha, especially from 10 to 1000 l/ha.

Preferred formulations can have the following compositions (weight %):

### Emulsifiable Concentrates:

active ingredient: 1 to 95%, preferably 60 to 90%
surface-active agent: 1 to 30%, preferably 5 to 20%
liquid carrier: 1 to 80%, preferably 1 to 35%

### Dusts:

active ingredient: 0.1 to 10%, preferably 0.1 to 5%
solid carrier: 99.9 to 90%, preferably 99.9 to 99%

### Suspension Concentrates:

active ingredient: 5 to 75%, preferably 10 to 50%
water: 94 to 24%, preferably 88 to 30%
surface-active agent: 1 to 40%, preferably 2 to 30%

### Wettable Powders:

active ingredient: 0.5 to 90%, preferably 1 to 80%
surface-active agent: 0.5 to 20%, preferably 1 to 15%
solid carrier: 5 to 95%, preferably 15 to 90%

### Granules:

active ingredient: 0.1 to 30%, preferably 0.1 to 15%
solid carrier: 99.5 to 70%, preferably 97 to 85%

The following Examples further illustrate, but do not limit, the invention.

| Wettable powders | a) | b) | c) |
|---|---|---|---|
| active ingredients | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The combination is thoroughly mixed with the adjuvants and the mixture is thoroughly ground in a suitable mill, affording wettable powders that can be diluted with water to give suspensions of the desired concentration.

| Powders for dry seed treatment | a) | b) | c) |
|---|---|---|---|
| active ingredients | 25% | 50% | 75% |
| light mineral oil | 5% | 5% | 5% |
| highly dispersed silicic acid | 5% | 5% | — |
| Kaolin | 65% | 40% | — |
| Talcum | — | | 20 |

The combination is thoroughly mixed with the adjuvants and the mixture is thoroughly ground in a suitable mill, affording powders that can be used directly for seed treatment.

Emulsifiable concentrate

| | |
|---|---|
| active ingredients | 10% |
| octylphenol polyethylene glycol ether | 3% |
| (4-5 mol of ethylene oxide) | |
| calcium dodecylbenzenesulfonate | 3% |
| castor oil polyglycol ether (35 mol of ethylene oxide) | 4% |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredients | 5% | 6% | 4% |
| Talcum | 95% | — | — |
| Kaolin | — | 94% | — |
| mineral filler | — | — | 96% |

Ready-for-use dusts are obtained by mixing the combination with the carrier and grinding the mixture in a suitable mill. Such powders can also be used for dry dressings for seed.

Extruder granules

| | |
|---|---|
| Active ingredients | 15% |
| sodium lignosulfonate | 2% |
| carboxymethylcellulose | 1% |
| Kaolin | 82% |

The combination is mixed and ground with the adjuvants, and the mixture is moistened with water. The mixture is extruded and then dried in a stream of air.

### Coated Granules

Active ingredients 8%
polyethylene glycol (mol. wt. 200) 3%

### Kaolin 89%

The finely ground combination is uniformly applied, in a mixer, to the kaolin moistened with polyethylene glycol. Non-dusty coated granules are obtained in this manner.

Suspension concentrate

| | |
|---|---|
| active ingredients | 40% |
| propylene glycol | 10% |
| nonylphenol polyethylene glycol ether (15 mol of ethylene oxide) | 6% |
| Sodium lignosulfonate | 10% |
| carboxymethylcellulose | 1% |
| silicone oil (in the form of a 75% emulsion in water) | 1% |
| Water | 32% |

She finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

Flowable concentrate for seed treatment

| | |
|---|---|
| active ingredients | 40% |
| propylene glycol | 5% |
| copolymer butanol PO/EO | 2% |
| Tristyrenephenole with 10-20 moles EO | 2% |
| 1,2-benzisothiazolin-3-one | 0.5% |
| (in the form of a 20% solution in water) | |
| monoazo-pigment calcium salt | 5% |
| Silicone oil (in the form of a 75% emulsion in water) | 0.2% |
| Water | 45.3% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

Slow Release Capsule Suspension

28 parts of the combination are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed. The obtained capsule suspension is stabilized by adding 0.25 parts of a thickener and 3 parts of a dispersing agent. The capsule suspension formulation contains 28% of the active ingredients. The medium capsule diameter is 8-15 microns. The resulting formulation is applied to seeds as an aqueous suspension in an apparatus suitable for that purpose.

The Examples which follow serve to illustrate the invention. The compounds of the invention can be distinguished from known compounds by virtue of greater efficacy at low application rates, which can be verified by the person skilled in the art using the experimental procedures outlined in the Examples, using lower application rates if necessary, for example 50 ppm, 12.5 ppm, 6 ppm, 3 ppm, 1.5 ppm or 0.8 ppm.

### EXAMPLE 1

This Example Illustrates the Preparation of (2E)-2-[4-(2,5-dichloro-3-thienyl)-1,3-dithiolan-2-ylidene]-2-imidazol-1-yl-acetonitrile (Compound I.h.25)

### a) Preparation of 2-chloro-1-(2,5-dichloro-3-thienyl)ethanol

Sodium borohydride (93 mg, 2.35 mmol) is added as a solid in portions at 0° C. to a solution of 2-chloro-1-(2,5-dichloro-3-thienyl)ethanone (2 g, 8.71 mmol) in methanol (12 mL). The reaction mixture is further stirred at room temperature for two hours and is quenched carefully with a saturated aqueous solution of ammonium chloride (15 mL). The aqueous mixture is extracted with methyl-ter-butylether. The combined organic phases are washed with brine, dried with sodium sulphate, filtered and are evaporated. Purification by chromatography on silica gel (heptanes/ethylacetate 20:1) give 2-chloro-1-(2,5-dichloro-3-thienyl)ethanol as pale yellow solid. ¹H-NMR (400 MHz, CDCl₃): δ=6.92 (s, 1H), 5.02 (td, J=3.7, 8.4 Hz, 1H), 3.74 (dd, J=3.7, 11.4 Hz, 1H), 3.62 (dd, J=8.4, 11.4 Hz, 1H), 2.64 (d, J=3.7 Hz, 1H). GC-MS (CI): m/z=232 (M+1).

### b) Preparation of [2-chloro-1-(2,5-dichloro-3-thienyl)ethyl] methanesulfonate

Methanesulfonyl chloride (102 μL, 1.3 mmol) is added at 0° C. to a solution of 2-chloro-1-(2,5-dichloro-3-thienyl)ethanol (300 mg, 1.3 mmol) and triethylamine (0.2 mL, 1.43 mmol) in ethyl acetate (2 mL). The resulting white suspension is stirred at 0° C. for 30 minutes and is filtered. The filtrate is diluted with H₂O (10 mL) and the biphasic mixture is decanted. The aqueous phase is extracted with ethyl acetate and the combined organic phases are washed with brine, dried with sodium sulphate, filtered and evaporated to give [2-chloro-1-(2,5-dichloro-3-thienyl)ethyl] methanesulfonate as pale yellow oil. ¹H-NMR (400 MHz, CDCl₃): δ=6.90 (s, 1H), 5.78 (dd, J=5.1, 7.7 Hz, 1H), 3.88-3.73 (m, 2H), 3.67 (s, 1H), 3.03 (s, 3H).

### c) Preparation of (2E)-2-[4-(2,5-dichloro-3-thienyl)-1,3-dithiolan-2-ylidene]-2-imidazol-1-yl-acetonitrile (Compound I.h.25)

A 25 mL flask is charged with powdered KOH (211 mg, 3.23 mmol), DMSO (3 mL), purged with Argon and is cooled at 10° C. with a water bath. A solution of 2-imidazol-1-ylacetonitrile (166 mg, 1.55 mmol) and CS₂ (0.195 mL, 3.23 mmol) in DMSO (3 mL) is then added slowly to give an orange mixture. The cooling bath is removed and the reaction is stirred at room temperature for 30 minutes. A solution of [2-chloro-1-(2,5-dichloro-3-thienyl)ethyl] methanesulfonate (400 mg, 1.29 mmol) in DMSO (2 mL) is then added dropwise. After 30 minutes, the reaction mixture is poured into H₂O (15 mL). The aqueous phase is extracted with dichloromethane, the combined organic phases are washed with brine, dried with Na₂SO₄, filtered and evaporated to give a crude pale yellow residue. Purification by chromatography on silica gel (heptanes/ethyl acetate, 5:1-1:1-1:3) afford (2E)-2-[4-(2,5-dichloro-3-thienyl)-1,3-dithiolan-2-ylidene]-2-imidazol-1-yl-acetonitrile (Compound I.h.25) as a white solid. Mp=117-119° C. ¹H-NMR (400 MHz, CDCl₃): δ=7.63 (s, 1H), 7.18 (s, 1H), 7.05 (t, J=1.3 Hz, 1H), 6.96 (s, 1H), 5.39 (dd, J=5.2, 8.7 Hz, 1H), 3.74 (dd, J=5.2, 11.9 Hz, 1H), 3.63 (dd, J=8.7, 11.9 Hz, 1H). MS (ESI): m/z=360, 362 (M+1).

Table 1 below illustrates examples of individual compounds of formula (I) according to the invention.

TABLE 1
individual compounds of formula (I) according to the invention

| Comp. | | | | | |
|---|---|---|---|---|---|
| No. | X | Y | R¹ | R² | R⁴ |
| 1 | NH | NH | H | H | H |
| 2 | NH | NH | CH₃ | H | H |
| 3 | NH | NH | CH₃ | CH₃ | H |
| 4 | NH | NH | H | H | CH₃ |
| 5 | NH | O | H | H | H |
| 6 | NH | O | CH₃ | H | H |
| 7 | NH | O | CH₃ | CH₃ | H |
| 8 | NH | O | H | H | CH₃ |
| 9 | NH | S | H | H | H |
| 10 | NH | S | CH₃ | H | H |
| 11 | NH | S | CH₃ | CH₃ | H |
| 12 | NH | S | H | H | CH₃ |
| 13 | O | NH | H | H | H |
| 14 | O | NH | CH₃ | H | H |
| 15 | O | NH | CH₃ | CH₃ | H |
| 16 | O | NH | H | H | CH₃ |
| 17 | O | O | H | H | H |
| 18 | O | O | CH₃ | H | H |
| 19 | O | O | CH₃ | CH₃ | H |
| 20 | O | O | H | H | CH₃ |
| 21 | S | NH | H | H | H |
| 22 | S | NH | CH₃ | H | H |
| 23 | S | NH | CH₃ | CH₃ | H |
| 24 | S | NH | H | H | CH₃ |
| 25 | S | S | H | H | H |
| 26 | S | S | CH₃ | H | H |
| 27 | S | S | CH₃ | CH₃ | H |
| 28 | S | S | H | H | CH₃ |

where
a) 28 compounds of formula (I.a):

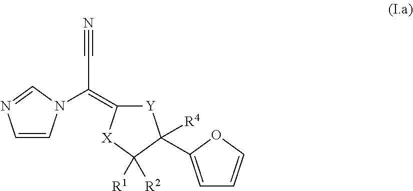

wherein X, Y, R¹, R² and R⁴ are as defined in Table 1.
b) 28 compounds of formula (I.b):

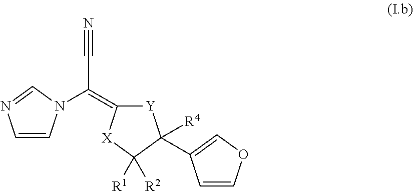

wherein X, Y, R¹, R² and R⁴ are as defined in Table 1.
c) 28 compounds of formula (I.c):

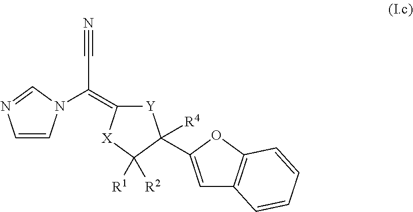

wherein X, Y, R¹, R² and R⁴ are as defined in Table 1.
d) 28 compounds of formula (I.d):

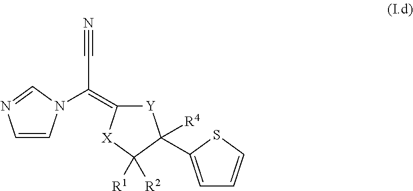

wherein X, Y, R¹, R² and R⁴ are as defined in Table 1.
e) 28 compounds of formula (I.e):

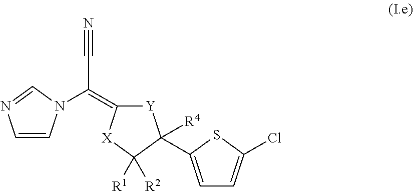

wherein X, Y, R¹, R² and R⁴ are as defined in Table 1.
f) 28 compounds of formula (I.f):

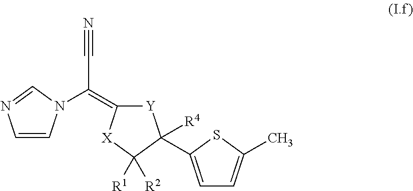

wherein X, Y, R¹, R² and R⁴ are as defined in Table 1.
g) 28 compounds of formula (I.g):

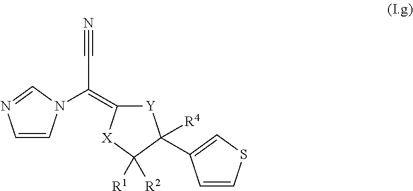

Wherein X, Y, R¹, R² and R⁴ are as defined in Table 1.
h) 28 compounds of formula (I.h):

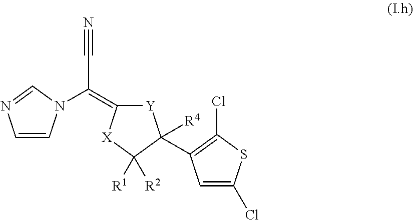

wherein X, Y, R¹, R² and R⁴ are as defined in Table 1.
i) 28 compounds of formula (I.i):

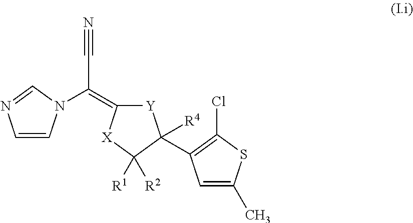

wherein X, Y, R¹, R² and R⁴ are as defined in Table 1.
j) 28 compounds of formula (I.j):

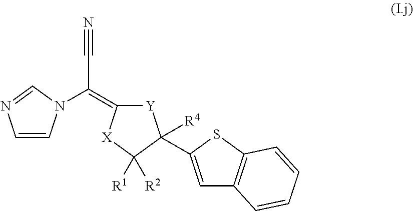

wherein X, Y, R¹, R² and R⁴ are as defined in Table 1.
k) 28 compounds of formula (I.k):

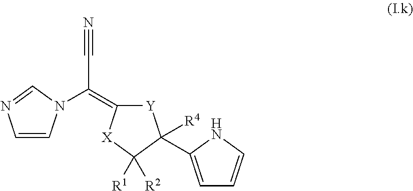

wherein X, Y, R¹, R² and R⁴ are as defined in Table 1.
m) 28 compounds of formula (I.m):

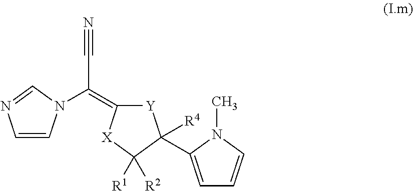

wherein X, Y, R¹, R² and R⁴ are as defined in Table 1.
n) 28 compounds of formula (I.n):

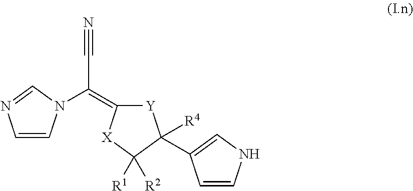

wherein X, Y, R¹, R² and R⁴ are as defined in Table 1.
o) 28 compounds of formula (I.o):

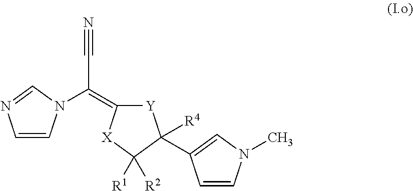

wherein X, Y, R¹, R² and R⁴ are as defined in Table 1.
p) 28 compounds of formula (I.p):

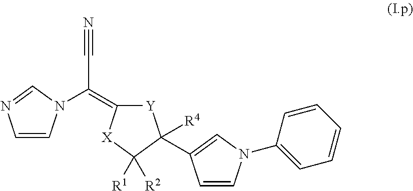

wherein X, Y, R¹, R² and R⁴ are as defined in Table 1.
q) 28 compounds of formula (I.q):

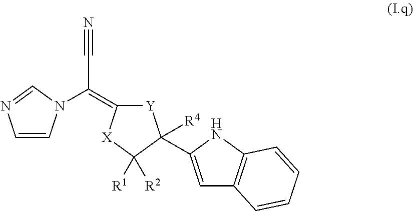

wherein X, Y, R¹, R² and R⁴ are as defined in Table 1.
r) 28 compounds of formula (I.r):

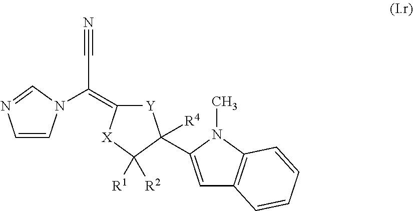

wherein X, Y, R¹, R² and R⁴ are as defined in Table 1.
s) 28 compounds of formula (I.s):

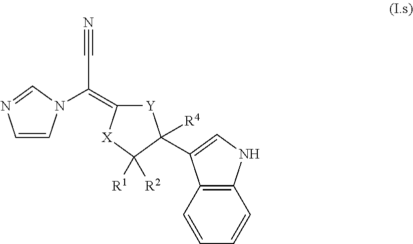

wherein X, Y, R¹, R² and R⁴ are as defined in Table 1.
t) 28 compounds of formula (I.t):

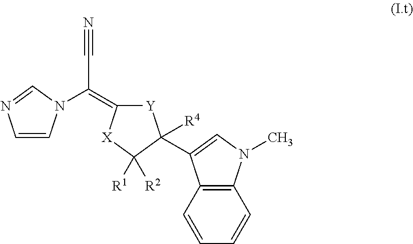

wherein X, Y, R¹, R² and R⁴ are as defined in Table 1.
u) 28 compounds of formula (I.u):

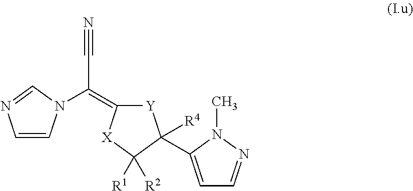

wherein X, Y, R¹, R² and R⁴ are as defined in Table 1.
v) 28 compounds of formula (I.v):

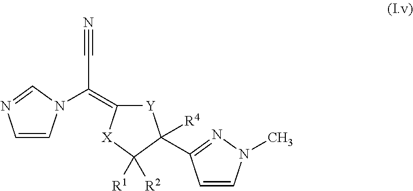

wherein X, Y, R¹, R² and R⁴ are as defined in Table 1.
w) 28 compounds of formula (I.w):

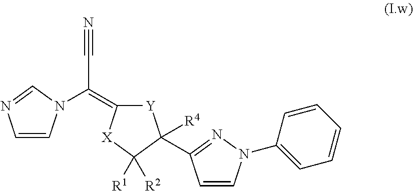

wherein X, Y, R¹, R² and R⁴ are as defined in Table 1.
x) 28 compounds of formula (I.x):

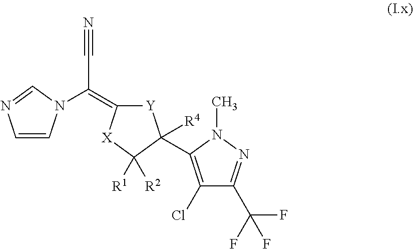

wherein X, Y, R¹, R² and R⁴ are as defined in Table 1.
y) 28 compounds of formula (I.y):

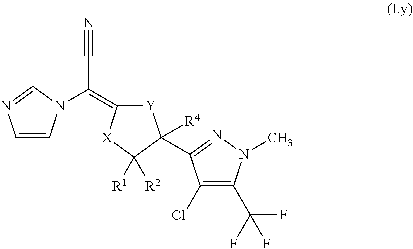

wherein X, Y, R¹, R² and R⁴ are as defined in Table 1.
z) 28 compounds of formula (I.z):

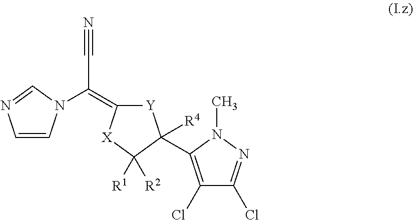

wherein X, Y, R¹, R² and R⁴ are as defined in Table 1.
aa) 28 compounds of formula (I.aa):

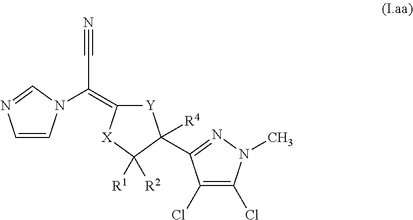

wherein X, Y, R¹, R² and R⁴ are as defined in Table 1.
ab) 28 compounds of formula (I.ab):

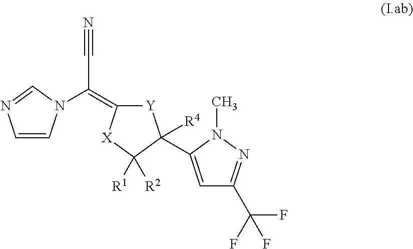

wherein X, Y, R¹, R² and R⁴ are as defined in Table 1.
ac) 28 compounds of formula (I.ac):

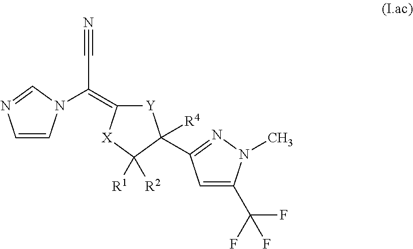

wherein X, Y, R¹, R² and R⁴ are as defined in Table 1.
ad) 28 compounds of formula (I.ad):

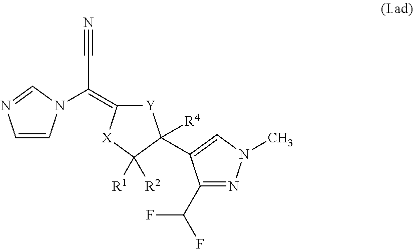

wherein X, Y, R¹, R² and R⁴ are as defined in Table 1.
ae) 28 compounds of formula (I.ae):

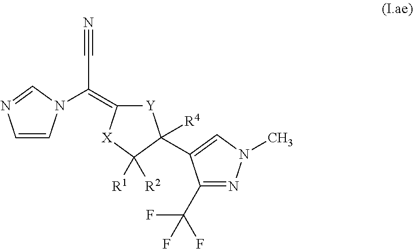

wherein X, Y, R¹, R² and R⁴ are as defined in Table 1.
af) 28 compounds of formula (I.af):

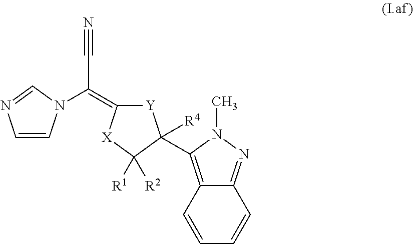

wherein X, Y, R¹, R² and R⁴ are as defined in Table 1.
ag) 28 compounds of formula (I.ag):

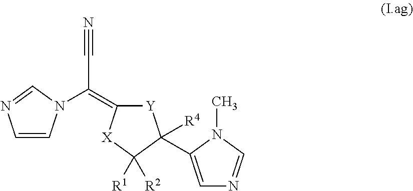

wherein X, Y, R¹, R² and R⁴ are as defined in Table 1.
ah) 28 compounds of formula (I.ah):

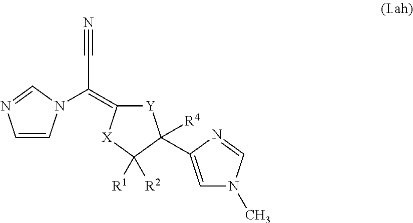

wherein X, Y, R¹, R² and R⁴ are as defined in Table 1.
ai) 28 compounds of formula (I.ai):

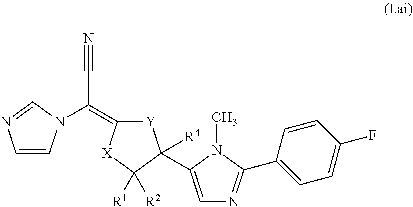

wherein X, Y, R¹, R² and R⁴ are as defined in Table 1.
aj) 28 compounds of formula (I.aj):

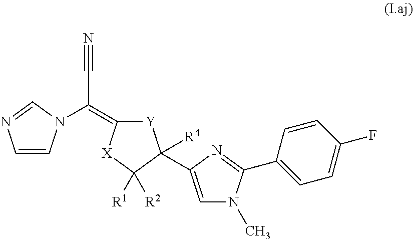

wherein X, Y, R¹, R² and R⁴ are as defined in Table 1.
ak) 28 compounds of formula (I.ak):

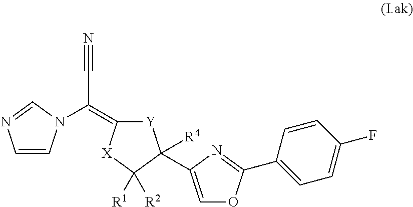

wherein X, Y, R¹, R² and R⁴ are as defined in Table 1.
am) 28 compounds of formula (I.am):

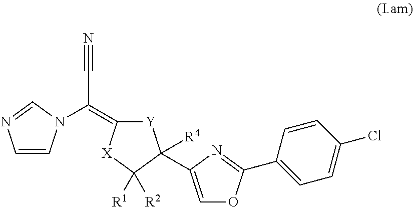

wherein X, Y, R¹, R² and R⁴ are as defined in Table 1.
an) 28 compounds of formula (I.an):

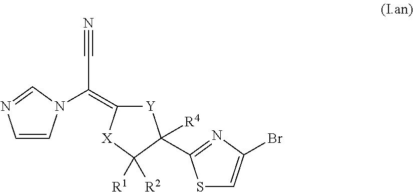

wherein X, Y, R¹, R² and R⁴ are as defined in Table 1.
ao) 28 compounds of formula (I.ao):

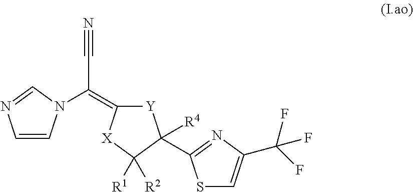

wherein X, Y, R¹, R² and R⁴ are as defined in Table 1.
ap) 28 compounds of formula (I.ap):

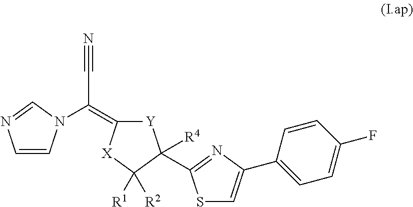

wherein X, Y, R¹, R² and R⁴ are as defined in Table 1.
aq) 28 compounds of formula (I.aq):

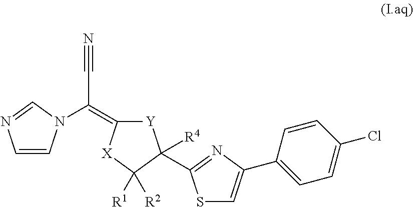

wherein X, Y, R¹, R² and R⁴ are as defined in Table 1.
ar) 28 compounds of formula (I.ar):

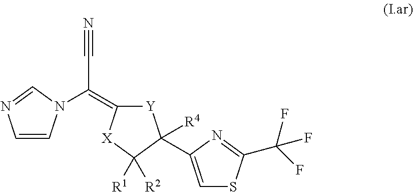

wherein X, Y, R¹, R² and R⁴ are as defined in Table 1.
as) 28 compounds of formula (I.as):

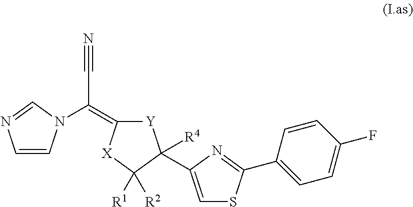

wherein X, Y, R¹, R² and R⁴ are as defined in Table 1.
at) 28 compounds of formula (I.at):

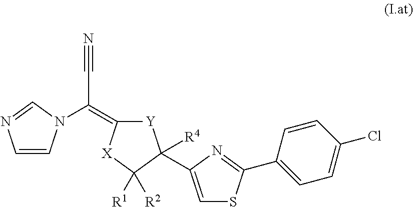

wherein X, Y, R¹, R² and R⁴ are as defined in Table 1.
au) 28 compounds of formula (I.au):

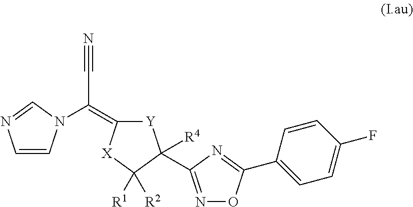

wherein X, Y, R¹, R² and R⁴ are as defined in Table 1.
av) 28 compounds of formula (I.av):

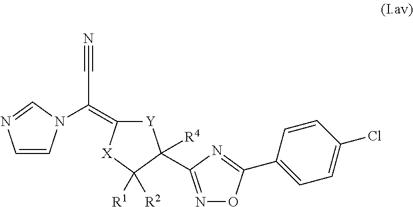

wherein X, Y, R¹, R² and R⁴ are as defined in Table 1.
aw) 28 compounds of formula (I.aw):

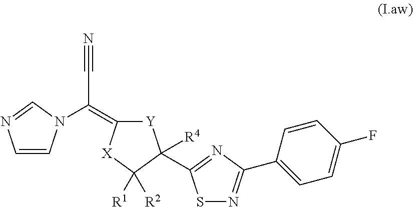

wherein X, Y, R¹, R² and R⁴ are as defined in Table 1.
ax) 28 compounds of formula (I.ax):

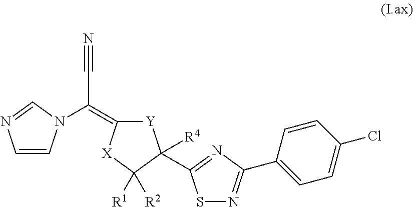

wherein X, Y, R¹, R² and R⁴ are as defined in Table 1.
ay) 28 compounds of formula (I.ay):

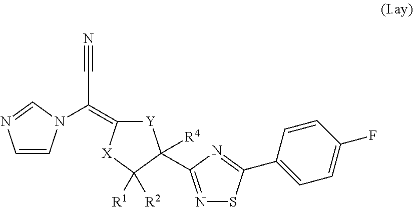

wherein X, Y, R¹, R² and R⁴ are as defined in Table 1.
az) 28 compounds of formula (I.az):

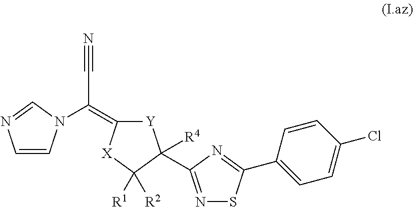

wherein X, Y, R¹, R² and R⁴ are as defined in Table 1.
ba) 28 compounds of formula (I.ba):

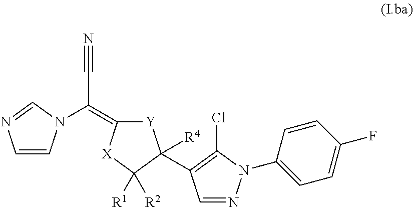

wherein X, Y, R¹, R² and R⁴ are as defined in Table 1.
bb) 28 compounds of formula (I.bb):

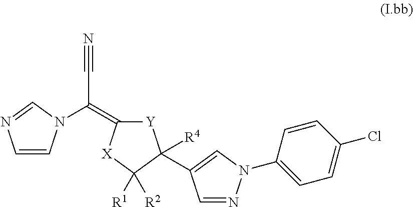

wherein X, Y, R¹, R² and R⁴ are as defined in Table 1.

Throughout this description, temperatures are given in degrees Celsius and “m.p.” means melting point. LC/MS means Liquid Chromatography Mass Spectroscopy and the description of the apparatus and the method is: (ACQUITY UPLC from Waters, Phenomenex Gemini C18, 3 μm particle size (3 micrometer particle size), 110 Angstrom, 30×3 mm column, 1.7 mL/min., 60° C., H₂O+0.05% HCOOH (95%)/CH₃CN/MeOH 4:1+0.04% HCOOH (5%)—2 min.—CH₃CN/MeOH 4:1+0.04% HCOOH (5%)—0.8 min., ACQUITY SQD Mass Spectrometer from Waters, ionization method: electrospray (ESI), Polarity: positive ions, Capillary (kV) 3.00, Cone (V) 20.00, Extractor (V) 3.00, Source Temperature (° C.) 150, Desolvation Temperature (° C.) 400, Cone Gas Flow (L/Hr) 60, Desolvation Gas Flow (L/Hr) 700)).

TABLE 2
Melting point and LC/MS data for compounds of Table 1

| | Melting | |
|---|---|---|
| Compound No. | point (° C.) | LC/MS |
| I.e.25 | | Rt = 0.86 min; MS: m/z = 326 (M + 1) |
| I.h.25 | 117-119 | |
| I.y.25 | | Rt = 0.89 min; MS: m/z = 392 (M + 1) |
| I.ad.25 | 147-148 | |
| I.am.25 | | Rt = 0.73 min; MS: m/z = 371 (M + 1) |
| I.ar.25 | 188-189 | |
| I.x.25 | 47-55 | |
| I.z.25 | | Rt = 0.83 min; MS: m/z = 360 (M + 1) |
| I.aa.25 | | Rt = 0.83 min; MS: m/z = 360 (M + 1) |
| I.ac.25 | | Rt = 0.81 min; MS: m/z = 358 (M + 1) |
| I.an.25 | | Rt = 0.73 min; MS: m/z = 373 (M + 1) |
| I.as.25 | 188-190 | |
| I.bb.25 | | Rt = 0.88 min; MS: m/z = 386 (M + 1) |

### BIOLOGICAL EXAMPLES

Alternaria solani/Tomato/Leaf Disc (Early Blight)

Tomato leaf disks cv. Baby are placed on agar in multiwell plates (24-well format) and sprayed with the formulated test compound diluted in water. The leaf disks are inoculated with a spore suspension of the fungus 2 days after application. The inoculated leaf disks are incubated at 23° C./21° C. (day/night) and 80% rh under a light regime of 12/12 h (light/dark) in a climate cabinet and the activity of a compound is assessed as percent disease control compared to untreated when an appropriate level of disease damage appears on untreated check disk leaf disks (5-7 days after application).

Compounds I.e.25, I.h.25, I.y.25, I.am.25, I.x.25, I.aa.25 and I.ac.25 at 200 ppm give at least 80% disease control in this test when compared to untreated control leaf disks under the same conditions, which show extensive disease development.

Blumeria raminis f. sp. tritici (Erysiphe raminis f. sp. tritici)/Wheat/Leaf Disc Preventative (Powdery Mildew on Wheat)

Wheat leaf segments cv. Kanzler are placed on agar in a multiwell plate (24-well format) and sprayed with the formulated test compound diluted in water. The leaf disks are inoculated by shaking powdery mildew infected plants above the test plates 1 day after application. The inoculated leaf disks are incubated at 20° C. and 60% rh under a light regime of 24 h darkness followed by 12 h light/12 h darkness in a climate chamber and the activity of a compound is assessed as percent disease control compared to untreated when an appropriate level of disease damage appears on untreated check leaf segments (6-8 days after application).

Compounds I.e.25, I.h.25, I.y.25, I.ad.25, I.am.25, I.x.25, I.z.25, I.aa.25, I.ac.25 and I.bb.25 at 200 ppm give at least 80% disease control in this test when compared to untreated control leaf disks under the same conditions, which show extensive disease development.

Botrvotinia fuckeliana (Botrvtis cinerea)/Liquid Culture (Gray Mould)

Conidia of the fungus from cryogenic storage are directly mixed into nutrient broth (Vogels broth). After placing a (DMSO) solution of test compound into a microtiter plate (96-well format), the nutrient broth containing the fungal spores is added. The test plates are incubated at 24° C. and the inhibition of growth is determined photometrically 3-4 days after application.

Compounds I.e.25, I.h.25, I.y.25, I.ad.25, I.am.25, I.x.25, I.z.25, I.aa.25 and I.ac.25 at 200 ppm give at least 80% disease control in this test when compared to untreated control leaf disks under the same conditions, which show extensive disease development.

Fusarium culmorum/Liquid Culture (Head Blight)

Conidia of the fungus from cryogenic storage are directly mixed into nutrient broth (PDB potato dextrose broth). After placing a (DMSO) solution of test compound into a microtiter plate (96-well format), the nutrient broth containing the fungal spores is added. The test plates are incubated at 24° C. and the inhibition of growth is determined photometrically 3-4 days after application.

Compounds I.e.25, I.h.25, I.y.25, I.ad.25, I.am.25, I.x.25, I.z.25 and I.aa.25 at 200 ppm give at least 80% disease control in this test when compared to untreated control leaf disks under the same conditions, which show extensive disease development.

Gaeumannomyces graminis/Liquid Culture (Take-all of Cereals)

Mycelial fragments of the fungus from cryogenic storage were directly mixed into nutrient broth (PDB potato dextrose broth). After placing a (DMSO) solution of test compound into a microtiter plate (96-well format), the nutrient broth containing the fungal spores is added. The test plates are incubated at 24° C. and the inhibition of growth is determined photometrically 4-5 days after application.

Compounds I.e.25, I.y.25, I.ad.25, I.am.25, I.z.25, I.aa.25 and I.ac.25 at 200 ppm give at least 80% disease control in this test when compared to untreated control leaf disks under the same conditions, which show extensive disease development.

Glomerella lacenarium (Colletotrichum lacenarium)/Liquid Culture (Anthracnose)

Conidia of the fungus from cryogenic storage are directly mixed into nutrient broth (PDB potato dextrose broth). After placing a (DMSO) solution of test compound into a microtiter plate (96-well format), the nutrient broth containing the fungal spores is added. The test plates are incubated at 24° C. and the inhibition of growth is measured photometrically 3-4 days after application.

Compounds I.e.25, I.h.25, I.ad.25, I.am.25, I.x.25, I.z.25, I.aa.25 and I.ac.25 at 200 ppm give at least 80% disease control in this test when compared to untreated control leaf disks under the same conditions, which show extensive disease development.

Magnaporthe grisea (Pyricularia oryzae)/Rice/Leaf Disc Preventative (Rice Blast)

Rice leaf segments cv. Ballila are placed on agar in a multiwell plate (24-well format) and sprayed with the formulated test compound diluted in water. The leaf segments are inoculated with a spore suspension of the fungus 2 days after application. The inoculated leaf segments are incubated at 22° C. and 80% rh under a light regime of 24 h darkness followed by 12 h light/12 h darkness in a climate cabinet and the activity of a compound is assessed as percent disease control compared to untreated when an appropriate level of disease damage appears in untreated check leaf segments (5-7 days after application).

Compound I.y.25 and I.z.25 at 200 ppm gives at least 80% disease control in this test when compared to untreated control leaf disks under the same conditions, which show extensive disease development.

Monocraphella nivalis (Microdochium nivale)/Liquid Culture (Foot Rot Cereals)

Conidia of the fungus from cryogenic storage are directly mixed into nutrient broth (PDB potato dextrose broth). After placing a (DMSO) solution of test compound into a microtiter plate (96-well format), the nutrient broth containing the fungal spores is added. The test plates are incubated at 24° C. and the inhibition of growth is determined photometrically 4-5 days after application.

Compounds I.e.25, I.h.25, I.y.25, I.ad.25, I.am.25, I.x.25, I.z.25, I.aa.25 and I.ac.25 at 200 ppm give at least 80% disease control in this test when compared to untreated control leaf disks under the same conditions, which show extensive disease development.

Mycosphaerella arachidis (Cercospora arachidicola)/Liquid Culture (Early Leaf Spot)

Conidia of the fungus from cryogenic storage are directly mixed into nutrient broth (PDB potato dextrose broth). After placing a (DMSO) solution of test compound into a microtiter plate (96-well format), the nutrient broth containing the fungal spores is added. The test plates are incubated at 24° C. and the inhibition of growth is determined photometrically 4-5 days after application.

Compounds I.e.25, I.h.25, I.y.25, I.ad.25, I.am.25, I.x.25, I.z.25, I.aa.25, I.ac.25 and I.as.25 at 200 ppm give at least 80% disease control in this test when compared to untreated control leaf disks under the same conditions, which show extensive disease development.

Mycosphaerella raminicola (Septoria tritici)/Liquid Culture (Septoria Blotch)

Conidia of the fungus from cryogenic storage are directly mixed into nutrient broth (PDB potato dextrose broth). After placing a (DMSO) solution of test compound into a microtiter plate (96-well format), the nutrient broth containing the fungal spores is added. The test plates are incubated at 24° C. and the inhibition of growth is determined photometrically 4-5 days after application.

Compounds I.h.25, I.y.25, I.ad.25, I.am.25, I.x.25, I.z.25, I.aa.25 and I.ac.25 at 200 ppm give at least 80% disease control in this test when compared to untreated control leaf disks under the same conditions, which show extensive disease development.

Pyrenophora teres/Barley/Leaf Disc Preventative (Net Blotch)

Barley leaf segments cv. Hasso are placed on agar in a multiwell plate (24-well format) and sprayed with the formulated test compound diluted in water. The leaf segments are inoculated with a spore suspension of the fungus 2 days after application. The inoculated leaf segments are incubated at 20° C. and 65% rh under a light regime of 12 h light/12 h darkness in a climate cabinet and the activity of a compound is assessed as disease control compared to untreated when an appropriate level of disease damage appears in untreated check leaf segments (5-7 days after application).

Compounds I.e.25, I.h.25, I.y.25, I.ad.25, I.am.25, I.x.25, I.z.25, I.aa.25, I.ac.25 and I.an.25 at 200 ppm give at least 80% disease control in this test when compared to untreated control leaf disks under the same conditions, which show extensive disease development.

Thanatephorus cucumeris (Rhizoctonia solani)/Liquid Culture (Foot Rot, Damping-Off)

Mycelia fragments of a newly grown liquid culture of the fungus are directly mixed into nutrient broth (PDB potato dextrose broth). After placing a (DMSO) solution of the test compounds into a microtiter plate (96-well format), the nutrient broth containing the fungal material is added. The test plates are incubated at 24° C. and the inhibition of growth is determined photometrically 3-4 days after application.

Compounds I.e.25, I.y.25, I.am.25, I.x.25, I.z.25 and I.ac.25 at 200 ppm give at least 80% disease control in this test when compared to untreated control leaf disks under the same conditions, which show extensive disease development.

1. A system for providing respiratory therapy, the system comprising:
a pressure generator configured to generate a pressurized flow of breathable gas for delivery to the airway of a subject;
an oscillator configured to cause high frequency pressure level oscillations in the pressurized flow of breathable gas;
one or more sensors configured to generate output signals conveying information related to one or more parameters of the gas; and
one or more physical computer processors configured by computer-readable instructions to (a) receive an input indicating a base expiratory pressure level and a base inspiratory pressure level; (b) control the pressure generator and the oscillator to generate the pressurized flow of breathable gas such that during exhalation the pressure level of the pressurized flow of breathable gas oscillates based on the received base expiratory pressure level and during inhalation the pressure level of the pressurized flow of breathable gas oscillates based on the received base inspiratory pressure level; and (c) detect expiratory flow limitation and to automatically adjust the base expiratory pressure level responsive to detecting expiratory flow limitation.

2. The system of claim 1, wherein the one or more physical computer processors are configured such that the high frequency pressure level oscillations are superimposed on the pressurized flow of breathable gas generated by the pressure generator.

3. The system of claim 1, wherein the oscillator includes one or more of a valve or an interrupter in a flow path of the pressurized flow of breathable gas.

4. The system of claim 1, wherein the one or more physical computer processors are configured such that detecting expiratory flow limitation is based on the output signals of the one or more sensors.

5. The system of claim 1, wherein the one or more physical computer processors are configured such that detecting expiratory flow limitation is based on one or more output signals of a pulse oximeter, an electromyogram, a pressure sensor, and/or a flow sensor.

6. A method of operation of a respiratory therapy system, the respiratory system comprising a pressure generator, an oscillator, one or more sensors, and one or more physical computer processors, the method comprising:
generating a pressurized flow of breathable gas with the pressure generator;
oscillating the pressurized flow of breathable gas at a night frequency;
generating output signals conveying information related to one or more parameters of the gas with the one or more sensors;
receiving an input indicating a base expiratory pressure level and a base inspiratory pressure level;
controlling the pressure generator and the oscillator to generate the pressurized flow of breathable gas such that during exhalation the pressure level of the pressurized flow of breathable gas oscillates based on the received base expiratory pressure level and during inhalation the pressure level of the pressurized flow of breathable gas oscillates based on the received base inspiratory pressure level; and
detecting expiratory flow limitation and automatically adjusting the base expiratory pressure level responsive to detecting the expiratory flow limitation.

7. The method of claim 6, wherein the high frequency oscillations are superimposed on the pressurized flow of breathable gas generated.

8. The method of claim 6, wherein the high frequency oscillations in the pressurized flow of breathable gas are caused by one or more of a valve or an interrupter in a flow path of the pressurized flow of breathable gas.

9. The method of claim 6, wherein detecting expiratory flow limitation is based on the output signal.

10. The method of claim 6, wherein detecting expiratory flow limitation is based on one or more output signals of a pulse oximeter, an electromyogram, a pressure sensor, and/or a flow sensor.

11. A system for providing respiratory therapy, the system comprising:
means for generating a pressurized flow of breathable gas for delivery to the airway of a subject;
means for causing high frequency pressure level oscillations in the pressurized flow of breathable gas;
means for generating output signals conveying information related to one or more parameters of the gas;
means for receiving an input indicating a base expiratory pressure level and a base inspiratory pressure level;
means for controlling the generation of the pressurized flow of breathable gas such that during exhalation the pressure level of the pressurized flow of breathable gas oscillates based on the received base expiratory pressure level and during inhalation the pressure level of the pressurized flow of breathable gas oscillates based on the received base inspiratory pressure level; and
means for detecting expiratory flow limitation and automatically adjusting the base expiratory pressure level responsive to detecting expiratory flow limitation.

12. The system of claim 11, wherein the high frequency pressure level oscillations are superimposed on the pressurized flow of breathable gas generated by the means for generating a pressurized flow of breathable gas.

13. The system of claim 11, wherein the means for causing high frequency pressure level oscillations include one or more of a valve or an interrupter in a flow path of the pressurized flow of breathable gas.

14. The system of claim 11, wherein the means for detecting expiratory flow limitation includes the output signals.

15. The system of claim 11, wherein the means for detecting expiratory flow limitation includes a pulse oximeter, an electromyogram, a pressure sensor, and/or a flow sensor.
